(19) United States
(12) Patent Application Publication
(10) Pub. No.: US 2026/0308094 A1
(43) Pub. Date: Oct. 8, 2026

(54) MIXED GUERBET REACTION OF ETHANOL AND METHANOL TO PRODUCE ISOBUTANOL

(71) Applicant: BASF SE, Ludwigshafen Am Rhein (DE)

(72) Inventors: Piyush INGALE, Heidelberg (DE); Ivana JEVTOVIKJ, Heidelberg (DE); Alexander CZAJA, Heidelberg (DE); Michael Ludwig LEJKOWSKI, Ludwigshafen am Rhein (DE); Christian GRUENANGER, Ludwigshafen am Rhein (DE); Stephan A. Schunk, Heidelberg (DE); Alois KINDLER, Ludwigshafen am Rhein (DE); Thomas HEIDEMANN, Ludwigshafen am Rhein (DE)

(21) Appl. No.: 19/482,852

(22) PCT Filed: May 8, 2024
(86) PCT No.: PCT/EP2024/062741

§ 371 Date: Nov. 10, 2025

(30) Foreign Application Priority Data
May 12, 2023 (EP) 23173203.3

(51) Int. Cl. C07C 29/34; B01J 6/00; B01J 23/72; B01J 27/236; B01J 37/04; B01J 37/18; C07C 31/12

(52) U.S. Cl. CPC C07C 29/34; B01J 6/001; B01J 23/72; B01J 27/236; B01J 37/04; B01J 37/18; C07C 31/12

(57) ABSTRACT

The invention relates to a method for producing iron-doped ruthenium/carbon carrier catalysts and to the use of same for selective liquid phase hydration of carbonyl compounds to obtain the corresponding alcohols, particularly for the hydration of citral to geraniol or nerol, or of citronellal to citronellol.

The present invention relates to a catalyst for condensing alcohols, which comprises support material in contact with copper as promoter, wherein
a) the support material comprises hydrotalcite-like compounds,
b) the proportion of copper promoter is in the range of from 0.05 to 5.0% by weight,
c) the support material has an Mg/Al ratio in the range of from 90/10 to 70/30, where the ratio is based on the weight of the respective oxides, wherein the catalyst may optionally comprise at least one further promoter element from the group consisting of Pt, Rh, Ru, Pd, Co, Ni, Pd, Cu, Ag and Au and the content of further promoter is in the range 0.01 to 2% by weight, preferably 0.01 to 1% by weight, with the proviso that the amount of copper promoter is greater than the amount of further promoter; and a process for condensing alcohols by bringing an alcohol-comprising feed gas stream, comprising an alcoholic component selected from the group consisting of C₁-C₅alcohols, especially at least two different alcoholic components from the group consisting of C₁-C₅alcohols, into contact with the catalyst. The high performance of the process of the invention in respect of the high conversion, the good yields and selectivities to the target compound, preferably iso-butanol, contribute, in particular, to an economical process having a high efficiency.

### PRIOR ART

Low carbon alcohols such as C₃-C₄ alcohols are important chemical intermediates used for the production of various solvents, plasticizers, polymers, lubricants, surfactants, and personal care products (J. Mück, K. Kocik, M. Hajek, Z. Tisler, K. Frohlich, A. Kasparek; Transition metals promoting Mg—Al mixed oxides for conversion of ethanol to butanol and other valuable products: Reaction pathways; Applied Catalysis A: General, pp 118380, 2021). Currently, synthesis of lower alcohols depends on the OXO synthesis process which involves the hydroformylation of olefins followed by hydrogenation (J. Sun, Y. Wang; Recent Advances in Catalytic Conversion of Ethanol to Chemicals, ACS Catalysis, pp 1078-1090, 2014). However, this process has many disadvantages including the difficulty in the separation of catalysts and products, the use of noble metals, and the harsh reaction condition of high pressure. Additionally, it is desirable to shift from the use of fossil-based raw materials toward biobased raw materials e.g. ethanol due to their reduced carbon footprint.

One of the promising pathways for the production of “green” isobutanol involves the Guerbet reaction. Guerbet reaction has been investigated for the last 100 years for the production of higher alcohol where primary or secondary alcohol possessing a methylene group at the α-position is condensed with itself or another alcohol (N. Egan, M. Kumbhalkar, J. Buchnan, J. Dumesci, G. Huber; Chemistries and processes for the conversion of ethanol into middle-distillate fuels, Nature Reviews, pp 223-249 (3), 2019).

The Guerbet condensation of ethanol and methanol involves the following four main steps
1. Dehydrogenation of ethanol and methanol to acetaldehyde and formaldehyde
2. Aldol condensation of acetaldehyde and formaldehyde to produce propionaldehyde
3. Further aldol condensation of propionaldehyde with formaldehyde to isobutyraldehyde
4. Hydrogenation of isobutyraldehyde to isobutanol

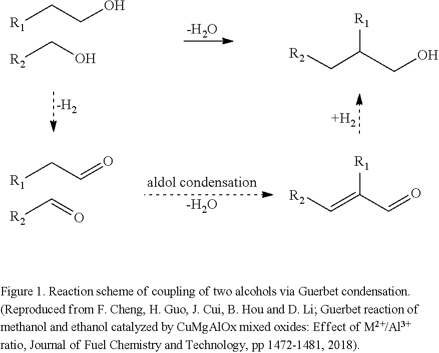

Various studies show the use of heterogeneous catalysts for the gas phase condensation of ethanol and methanol to either propanol or isobutanol. C. Carlini, M. Di Girolamo, A. Macinai, M. Marchionna, M. Noviello, A. Galletti, G. Sbrana; Selective synthesis of isobutanol by means of the Guerbet reaction: Part 2. Reaction of methanol/ethanol and methanol/ethanol/n-propanol mixtures over copper based/MeONa catalytic systems; Journal of Molecular Catalysis A: Chemical, pp 137-146, 2003 investigated the Guerbet condensation of ethanol/methanol and ethanol/methanol/propanol mixtures using sodium methoxide (MeONa) as a soluble basic component and copper-based heterogeneous catalysts.

J. Suarez, B. Subrmanim, R. Chaudhari; Vapor-phase methanol and ethanol coupling reactions on CuMgAl mixed metal oxides, Applied Catalysis A: General, pp 234-246, 2013 studied the effect of the catalyst composition of CuMgAlOₓ with varying Cu content on methanol and ethanol coupling reactions. Their observations indicated that MgAlOₓ without Cu deactivated rapidly while producing mainly C3+ alcohols at low alcohol conversions. Whereas in CuMgAlOx, C—C coupling (C3+ aldehydes, alcohols, and esters), non-C—C coupling (e.g., acetaldehyde, methyl formate, methyl acetate, ethyl acetate), and decomposition products (i.e., COx) were observed at significantly larger methanol and ethanol conversions and with minimal catalyst deactivation. Furthermore, D. Stošić, F. Hosoglu, S. Bennici, A. Travert, M. Capron, F. Dumeignil, J.-L. Couturier, J.-L. Duboise, A. Aurou; Methanol and ethanol reactivity in the presence of hydrotalcites with Mg/Al ratios varying from 2 to 7, Catalysis Communications, pp 14-18, 2017 confirmed the correlation between acidic and basic properties of hydrotalcite materials (Mg:Al ratio) to the reactivity of Guerbet condensation of ethanol and methanol. The highest alcohol conversions were reported for materials with the highest number of acidic and basic sites yielding high propanol and isobutanol selectivity. Cheng et. al., Journal of Fuel Chemistry and Technology, pp 1472-1481, 2018 showed the role of Cu species and alkalinity of CuMgAlOx mixed oxide catalysts towards Guerbet condensation of ethanol and methanol. Their results showed that the conversions of methanol and ethanol are strongly related to the surface areas of exposed Cu species and altered basicity.

U.S. Pat. No. 5,095,156 disclosed the conversion of methanol and ethanol over MgO or MgO admixed with charcoal. The reaction was carried out between sub-atmospheric pressure to up to 10 bar yielding the majority C3 and C4 alcohols in addition with COₓ and paraffin.

U.S. Pat. No. 5,559,275 relates to a process for the production of branched C4+ oxygenates from lower alcohols such as methanol, ethanol, propanol and mixtures thereof. The process comprises contacting the lower alcohols with a solid catalyst comprising a mixed metal oxide support having components selected from the group consisting of oxides of zinc, magnesium, zirconia, titanium, manganese, chromium, and lanthanides, and an activation metal selected from the group consisting of Group VIII metal, Group IB metals, and mixtures thereof.

U.S. Pat. No. 5,770,541 discloses a catalyst for the synthesis of isobutanol. In the catalyst, a noble metal is present on a support and the support has a first phase composed of crystallites of a mixed oxide comprising zirconium, manganese, zinc and a second phase having zirconium-doped hetaerolite particles comprising manganese and zinc. In addition, the support can also comprise a third phase composed of manganese, zinc and zirconium. A characteristic of the catalyst is that the crystallites of the first phase have a size in the range from 40 Å to 100 Å, the crystallites of the second phase have a size in the range from 200 Å to greater than 2000 Å and the crystallites of the third phase have a size in the range from 1000 Å to greater than 4000 Å.

U.S. Pat. No. 7,705,192 disclosed a process for making an isobutanol-containing product by contacting a reactant comprising ethanol and methanol over a catalyst wherein said reaction temperature is from about 200° C. to about 500° C. and said pressure is from about 0.1 MPa to about 20.7 MPa. The catalyst was derived from hydrotalcite of formula (M²⁺ 1-xM³⁺x (OH)₂)(An-x/n). yH₂O wherein M²⁺ is divalent Mg, or a combination of divalent Mg and at least one divalent member selected from the group consisting of Zn, Ni, Pd, Pt, Co, Fe, and Cu; M^{3+ is trivalent Al, or a combination of trivalent Al and at least one trivalent member selected from the group consisting of Fe and Cr.}

In WO2009/097310A1 discloses a process for the catalytic reaction of ethanol in the presence of hydrogen. The process is based on catalysts which comprise thermally decomposed hydrotalcite, and the catalyst synthesis is carried out in the presence of EDTA (ethylenediaminetetraacetic acid) as complexing agent. The examples disclose the production of catalysts having cobalt as active metal.

According to WO2009/026506A1 hydrotalcites containing the anion of ethylenediaminetetraacetic acid are partially or fully thermally decomposed to provide catalysts useful for the conversion of ethanol and methanol to a reaction product comprising isobutanol.

CN105562046B discloses flower-shaped mesoporous hydroxyl Apatite catalysts with Ca/P/Sr/Cu active metal oxides to produce propyl alcohol through Guerbet condensation of ethanol and methanol.

WO2012035772A1 (EP2616418A1) relates to a method for producing an alcohol by a Guerbet reaction, wherein the reaction is performed in a gas phase and at a total pressure of less than 1 atm, using one or more raw material alcohols. The method involves the use of a basic catalyst which preferably comprises an apatite structure compound, such as, for example, calcium hydroxyapatite, strontium hydroxyapatite, hydrotalcite, MgO, Mg(OH)₂, and alkali metal supported-zeolite.

CN105562046B relates to a catalyst for condensing methanol and ethanol to prepare propyl alcohol and butanol. The catalyst uses flower-shaped mesoporous hydroxyapatite as the carrier, and oxide is loaded on the surface. The oxides are copper oxide and strontium oxide. The mole ratio of Ca to P to Sr to Cu is 1.65-1.9:1:0.019-0.089:0.018-0.067.

C. Carlini et al. report carrying out the Guerbet condensation using bifunctional catalysts based on magnesium- and aluminum-comprising mixed oxides (see C. Carlini et al., Journal of Molecular Catalysis A: Chemical 232 (2005) 13-20). The catalysts can comprise the elements Pd, Rh, Ni and Cu as active metals. Furthermore, it is stated that the magnesium- and aluminum-comprising mixed oxides used for producing the catalysts have a hydrotalcite structure. Marcu et al. describe the conversion of ethanol into butanol by means of mixed oxide catalysts which are produced from magnesium- and aluminum-comprising hydroxides having a double layer structure (see loan-Cezar Marcu, Nathalie Tanchoux, Francois Fajula, Didier Tichit, Catal. Lett (2013) 143, p. 23-30). The catalysts can comprise the metals of the group consisting of Pd, Ag, Mn, Fe, Cu, Sm, Yb as active metals.

A publication by Di Cosimo et al. describes the use of catalysts based on magnesium- and aluminum-comprising mixed oxides for the condensation of alcohols (see J. I. Di Cosimo, Journal of Catalysis (2000) 190, p. 261-275). The publication presents a fundamental study on the influence of the support oxides on the catalytic reaction processes in the dimerization reaction.

Carlini et al., Journal of Molecular Catalysis A: Chemical 232 (2005) pages 13-20 report the synthesis of isobutyl alcohol from methanol and n-propanol through the guerbet condensation using bifunctional heterogeneous systems based on a dehydrogenating/hydrogenating metal (Pd, Rh, Ni or Cu) and a basic Mg—Al mixed oxide derived from hydrotalcite-type (HT) precursors.

F. Storgards et al., Topics in Catalysis, 61 (2018), pages 1888-1900 disclose (Table 1) a catalyst comprising 5 wt % Cu supported on hydrotalcite (Mg/Al ratio 2.25). The catalyst was prepared by coprecipitation of the corresponding metal nitrates. The amounts of Mg and Al were selected to give the same Mg/Al ratio as in the commercial HT, ca. 2.25. It was the aim to enhance the one-pot production of Guerbet alcohol (2BO (=2-butyl-2-octenal) from hexanol with the aid of heterogeneous catalysis.

K. A. Goulas et al., Journal of the American Chemical Society 138 (2016) pages 6805-6812 reports a bimetallic catalyst (Pd—Cu) supported on hydrotalcite in the 1-octanol Guerbet reaction.

US2023/037136 relates to a method for producing a Guerbet alcohol, comprising reacting a raw material alcohol having 8 or more and 22 or less carbon atoms, in the presence of a catalyst (A) containing a first component, a second component, and a third component below:
first component: copper,
5 second component: one kind selected from the group consisting of cobalt, nickel, molybdenum, and rhenium, and
third component: at least one kind selected from the group consisting of titanium, iron, zinc, yttrium, zirconium, niobium, molybdenum, cerium, samarium, tantalum, tungsten, rhenium, and gold, and are different from the element selected as the second component.

WO201518793A1 (EP3030346A1) relates to a catalyst for condensing alcohols, which comprises partially or fully thermally decomposed support material in contact with iridium and/or ruthenium as promoter, wherein
a) the partially or fully decomposed support material comprises hydrotalcite-like compounds, preferably hydrotalcite, and/or precursor material of hydrotalcite-like compounds, preferably hydrotalcite precursor material, as starting material,
b) the proportion of Ru and/or Ir promoter is in the range 0.05-4% by weight, preferably 0.1-3.5% by weight and particularly preferably in the range 0.2-3.0% by weight and the average particle size of the promoter particles is <100 nm, preferably <75 nm, more preferably <50 nm and particularly preferably <20 nm,
c) the thermally decomposed support material has an Mg/Al ratio in the range 90/10-40/60, preferably from 90/10 to 70/30, where the ratio is based on the weight of the respective oxides; and a process for condensing alcohols by bringing an alcohol-comprising feed (gas) stream comprising an alcohol selected from the group consisting of C₁-C₅-alcohols into contact with the catalyst, wherein
(i) the process temperature is in the range from 200 to 450° C., preferably 250° C.-400° C.,
(ii) the process pressure is in the range 0.05-60 bar, more preferably 0.1-40 bar, particularly preferably 0.5-10 bar, even more preferably in the range 1-5 bar,
(iii) the alcohol content of the feed (gas) stream is in the range 0.5-90% by volume, preferably in the range 0.5-70% by volume and more preferably in the range 0.5-50% by volume,
(iv) the feed (gas) stream has a GHSV in the range 500-5000 h⁻¹, preferably in the range 1000-4000 h⁻¹, particularly preferably in the range 1000-2500 h⁻¹.

It is an object of the invention to provide an improved process for condensing alcohols in the gas phase. The process should, in particular, also be able to be utilized for the preparation of iso-butanol from methanol and ethanol. In addition, the process should also be suitable for preparing branched and unbranched C₃-C₁₀-alcohols. Furthermore, a catalyst suitable for the process of the invention should be provided.

### Catalyst of the Invention

The objects mentioned here are achieved by discovery of a catalyst for condensing alcohols, which comprises (partially or fully thermally decomposed) support material in contact with copper as promoter, wherein
a) the (partially or fully decomposed) support material comprises hydrotalcite-like compounds, preferably hydrotalcite, and/or precursor material of hydrotalcite-like compounds, preferably hydrotalcite precursor material, as starting material,
b) the proportion of copper promoter is in the range 0.05 to 20% by weight, preferably 0.05 to 10% by weight and particularly preferably in the range of from 0.05 to 5.0% by weight,
c) the (thermally decomposed) support material has an Mg/Al ratio in the range 90/10-40/60, preferably from 90/10 to 70/30, where the ratio is based on the weight of the respective oxides, wherein the catalyst may optionally comprise at least one further promoter element from the group consisting of Pt, Rh, Ru, Pd, Co, Ni, Pd, Cu, Ag and Au and the content of further promoter is in the range 0.01 to 2% by weight, preferably 0.01 to 1% by weight, with the proviso that the amount of copper promoter is greater than the amount of further promoter. The support material and the promotor(s) add up to 100% by weight.

The copper comprising promoter particles are embedded in the matrix of the partially or fully thermally decomposed support material and are highly disperse, which has a favorable effect on the properties of the material and thus represents an important aspect of the invention. The catalysts of the invention described here have a high sintering resistance while the process of the invention is carried out.

In particular, copper in the combination according to the invention has been found to be a very particularly suitable promoter element, which was not to be expected in this form. Thus, a particularly suitable catalyst which displays totally extraordinary performance properties in the process of the invention has been able to be found on the basis of copper. The catalyst of the invention is thus superior to the catalysts and processes known from the prior art.

It is also worth mentioning that the content of copper-comprising promoter is very low and very good performance properties in the process of the invention for condensing alcohols are nevertheless achieved using the catalyst of the invention.

In addition, the catalyst of the invention can comprise at least one further promoter element. The further promoter elements comprise elements from the group consisting of Pt, Rh, Ru, Pd, Co, Ni, Pd, Cu, Ag and Au, with the content of further promoter elements or of further promoter element preferably being in the range 0.01 to 1% by weight, more preferably 0.01 to 0.5% by weight. The support material and the promotor(s) add up to 100% by weight.

In a preferred embodiment the catalyst comprises at least one further promoter element from the group consisting of Ru and Ir and the content of further promoter is in the range 0.01 to 2% by weight, preferably 0.01 to 1% by weight. In said embodiment the proportion of copper promoter is in the range 0.05 to 5.0%, especially 0.05 to 2.0% by weight and Ir, or Ru are preferably present in an amount of 0.05 to 0.15% by weight, more preferably about 0.1% by weight.

In a more preferred embodiment the proportion of copper promoter is in the range 0.05 to 5.0% by weight, especially 0.05 to 2.0% by weight and no further promoter element is present.

Hydrotalcite and/or Hydrotalcite Precursor Materials or Hydrotalcite-Like Compounds as Starting Material(s)

The term “hydrotalcite-like compound” is used as a generic term encompassing all compounds having the same basic structure as hydrotalcite as such. Hydrotalcite has the formula Mg₄Al₂(OH)₁₂CO₃·4H₂O.

The term “precursor material of hydrotalcite-like compound” is used to mean starting materials used for the production of hydrotalcite-like compounds”.

Hydrotalcite-like compounds are mixed hydroxides of divalent and trivalent metals, which are made up of polycations and have a layer structure. Hydrotalcite-like compounds are also referred to in the literature as anionic clays, layered double hydroxides (=LDHs), Feitknecht compounds or double layer structures. When starting from the parent compound hydrotalcite of the formula Mg₄Al₂(OH)₁₂CO₃·4H₂O, Al³⁺ can be partially or completely replaced by trivalent metal cations of similar size such as, for example, Ga³⁺, Fe³⁺, Mn³⁺ and Cr³⁺ and independently Mg²⁺ can be replaced by divalent cations of similar size, such as, for example, Mg²⁺, Zn²⁺, Fe²⁺ and Mn²⁺.

In a preferred embodiment, the hydrotalcite-like compound consists of hydrotalcite. The hydrotalcites used for the process of the invention preferably comprise magnesium as divalent metal and aluminum as trivalent metal. The metals of the hydrotalcites used preferably consist predominantly of magnesium and aluminum.

Another characteristic is that the hydrotalcite-like compounds and the promotor source are very intimately mixed.

Such mixing can, for example, be achieved by physical mixing of hydrotalcite-like and aluminum hydroxide-comprising powders, for example by powder mixing in suitable technical apparatuses such as mixers. Such intimate mixing processes are known to those skilled in the art. A further possibility is to mix the hydrotalcite-like powder and the aluminum hydroxide-comprising powder in suitable dispersion media. As dispersion media, it is possible to use, for example, water, alcohols such as methanol, ethanol, propanol, butanol, ethylene glycol and/or butanediol and ketones such as acetone or methyl ethyl ketone. It is also possible for the dispersion media to be present as mixtures and comprise surface-active agents such as surfactants. Examples of such surfactants are, inter alia, polyethylene glycols, mersolates, carboxylates, long-chain ammonium compounds such as CTAB.

The process of the invention is preferably carried out using hydrotalcites or hydrotalcite-like compounds having a particularly high purity. The process for preparing these hydrotalcite-like compounds as are particularly preferably used in the process of the invention is disclosed in DE19503522A1.

According to DE19503522A1 the hydrotalcites or hydrotalcite-like compounds are formed by hydrolysis of metal alkoxides by means of water and subsequent drying of the hydrolysis products which have separated out as precipitate. The metal alkoxides are formed by reaction of monovalent, divalent and/or trivalent alcohols with one or more divalent metals and/or one or more trivalent metals. The water used for the hydrolysis preferably comprises water-soluble anions selected from the group consisting of hydroxide anions, organic anions, in particular alkoxides, alkyl ether sulfates, aryl ether sulfates and/or glycol ether sulfates, and/or inorganic anions, in particular carbonate, hydrogencarbonate, chloride, nitrate, sulfate and/or polyoxometalate anions. Ammonium is preferably used as counterion.

Suitable starting materials are commercially available hydrotalcites which can be procured, for example, under the name Pural MG from Sasol. Otherwise, the hydrotalcites or the hydrotalcite precursor materials can also be prepared by a person skilled in the art, for example via the precipitation products of metal salts which are precipitated in the appropriate stoichiometric ratios and are converted by thermal treatment into the desired structures/desired structure.

The preferred starting material is a mixture of magnesium and aluminum, either as oxide mixture or as mixture of oxides and elemental metals.

Furthermore, it is also possible to synthesize hydrotalcite and/or hydrotalcite precursor material as starting material (the support precursor) from a finely divided aluminum oxide and aluminum hydroxide mixture, for example the commercially available material Disperal (Sasol), and a suitable magnesium source. As magnesium source, it is possible to use, for example, any water-soluble salt, with magnesium nitrates being particularly useful. It has also been found that the magnesium cations or atoms can be partly replaced by other cations or atoms while maintaining the catalytic properties of the material. For example, magnesium can be replaced by other divalent alkaline earth metals, preferably calcium. In a further embodiment, it is possible to bring a mixture of calcium oxide and aluminum oxide into contact with a magnesium-comprising compound. Here, the calcium oxide and aluminum oxide should be present in a ratio of from 90/10 to 40/60, preferably from 90/10 to 70/30. As magnesium-comprising compound, it is possible to use, for example, any water-soluble salt, with magnesium nitrates being particularly useful. The ratio here is the weight ratio of the oxides.

The suitable starting material (i.e. support precursor) can also be synthesized by a precipitation process. For this purpose, an aqueous solution of the mixture of magnesium- and aluminum-comprising salts is prepared in such a way that magnesium and aluminum cations are present in the ratio described below. All water-soluble salts are suitable for the preparation of the salt mixture, with the nitrates having been found to be particularly useful. Furthermore, it is also possible to synthesize the starting material (or the support precursor) from a finely divided aluminum oxide and aluminum hydroxide mixture, for example the commercially available material Disperal (Sasol) and a suitable magnesium source. As magnesium source, it is possible to use, for example, any water-soluble salt, with magnesium nitrates being particularly useful.

The introduction of the promoter elements via suitable starting materials is also of importance. The background is that the homogeneity of the dispersion in the solution plays a critical role in the dispersion and distribution of the particles present in the catalyst of the invention. Suitable starting materials for precipitating or obtaining the Cu comprising promoters are: halides or other inorganic salts such as sulfates and nitrates, also acetates, acetylacetonates and oxalates, and also olefin complexes, complexes with pyridine or other amine ligands, carbonyl complexes and finally also complexes with phosphanes, phosphides and phosphates.

As examples of compounds which can be used for the catalyst synthesis, mention may be made of the following:

Examples of Cu-comprising compounds: Cu(NO₃)₂, Cu₂SO₄, CuCl₂, Cu(OAc)₂ and Cu(O₂C₅H₇)₂. Examples of Ru-comprising compounds: Ru(NO)(NO₃)₃, Ru(NO₃)₃, RuCl₃, RuCl₃·xH₂0, Ru₃(CO)₁₂, Ru(OAc)₃, Ru(acac)₃, Ru(CO)₂(OAc)₂, RuCl₂(cod), [RuCl₂(C₆H₆)]₂, Ru(Cp)Cl(PPh₃)₂, [Cp*RuCl]₄, RuHCl(PPh₃)₃, RuH₂(CO)(PPh₃)₃, RuCl₂(PPh₃)₃ and RuH₂(PPh₃)₄. Examples of iridium-comprising compounds: IrCl₃, IrCl₃·xH₂0, IrCl₄·xH₂O, IrO₂, Ir(OAc)₃, Ir(acac)₃, Ir(cod)(acac), IrH(CO)(PPh₃)₃, [Cp*IrCl₂]₂, [IrCl(cod)]₂ and Ir₄(CO)₁₂.

### Details of the Production of the Catalyst of the Invention

The invention also provides a catalyst for condensing alcohols, which comprises (partially or fully thermally decomposed) support material in contact with Cu as promoter, where the catalyst of the invention can be produced by the following steps:
d) support material comprising a hydrotalcite-like compound, preferably hydrotalcite-comprising support material, and/or precursor material of a hydrotalcite-like compound, preferably hydrotalcite precursor material, is brought into contact with a promoter source,
e) an intimate mixture of support material comprising a hydrotalcite-like compound, preferably hydrotalcite-comprising support material, and/or precursor material of a hydrotalcite-like compound, preferably hydrotalcite precursor material, and the promoter source is produced,
f) the intimate mixture of support material comprising a hydrotalcite-like compound, preferably hydrotalcite-comprising support material, and/or precursor material of a hydrotalcite-like compound, preferably hydrotalcite precursor material, and the promoter source is treated thermally, with the thermal treatment comprising a calcination process at a temperature in the range 200-1000° C., preferably 200-900° C. and particularly preferably 200-850° C.

The catalyst of the invention is based on a partially or fully thermally decomposed support material, where the thermal decomposition as per step c) of the production process is based on a calcination treatment. Also of central importance here is that the contacting with the promoter source as per step a) and the intimate mixing of the promoter source with the precursor material of the support material as per step b) always has to be carried out before the calcination treatment in step c).

In a preferred embodiment, the production process can comprise a multistage calcination process with a treatment at a first temperature level and a treatment at a second temperature level.

### Shaping and Molding of the Catalyst

The shaping step may be, for example, tableting, extrusion, spray drying, granulation or similar processes which are known to those skilled in the art. An extrusion or tableting process may be used for producing the catalyst. The shaped bodies obtained here can be obtained in various sizes and shapes. For example, pellets have dimensions of 3 mm in length and 6 mm in diameter, 5 mm in length and 5 mm in diameter or 5 mm in length and 8 mm in diameter.

The compacting stage may also be carried out a number of times in succession in order to increase the efficiency. This compaction may be carried out on pulverulent starting materials using a roller compacter. Here, small amounts of water can also be added to the starting materials in order to convert the powder into a kneadable paste.

### Calcination Process

In a preferred embodiment of the production process, the calcination process comprises multistage heating of the catalyst at least two different temperature levels.

The use of preferred heating rates during heating-up of the samples is advantageous in order to avoid local exothermic combustion processes and associated sintering processes within the catalyst. In addition, it can be preferred that the samples are subjected to pre-drying in order to commence the calcination process with samples having a water content of not more than 50% by weight.

In a preferred calcination procedure, the sample impregnated with promoter species is, for example, calcined in a two-stage process in which the thermal treatment of the sample in the first calcination stage is carried out in the range from 200° C. to 300° C., preferably from 250° C. to 300° C., and the thermal treatment of the sample in the second calcination stage is carried out in the range from 350° C. to 1000° C., preferably from 400° C. to 900° C. and particularly preferably from 400° C. to 850° C. Furthermore, heating the impregnated sample at a controlled heating rate to the target temperatures can be preferred, with a preferred heating rate providing a temperature rise of 0.5-3.0 K/min, preferably 0.5-1.5 K/min. The calcination is preferably carried out under an air atmosphere, with the air more preferably being passed over the sample at a flow rate of 3-10 l/min, more preferably from 5 to 8 l/min. The amount of air passed through the furnace depends on the respective furnace volume.

The catalyst of the invention may have a high specific surface area, which can be determined by means of nitrogen sorption.

In a particularly preferred embodiment the present invention is directed to a catalyst for condensing alcohols, which comprises support material in contact with copper as promoter, wherein
a) the support material comprises hydrotalcite,
b) the proportion of copper promoter is in the range of from 0.05 to 2.0% by weight,
c) the support material has an Mg/Al ratio in the range of 90/10-40/60, preferably from 90/10 to 70/30, where the ratio is based on the weight of the respective oxides, wherein the catalyst comprises no further promoter element.

In another particularly preferred embodiment the present invention is directed to a catalyst for condensing alcohols, which comprises support material in contact with copper as promoter, wherein
a) the support material comprises hydrotalcite,
b) the proportion of copper promoter is in the range of from 0.05 to 5.0% by weight, especially 0.05 to 2.0% by weight,
c) the support material has an Mg/Al ratio in the range of 90/10-40/60, preferably from 90/10 to 70/30, where the ratio is based on the weight of the respective oxides, wherein the catalyst comprises at least one further promoter element from the group consisting of Ru and Ir and the content of further promoter is in the range of from 0.01 to 1.0% by weight, preferably 0.05 to 0.15% by weight, with the proviso that the amount of copper promoter is greater than the amount of further promoter.

The catalyst for condensing alcohols according to the particularly preferred embodiments is obtainable by a process comprising the following steps:
d) support material comprising hydrotalcite and/or precursor material of a hydrotalcite precursor material is brought into contact with a promoter source,
e) an intimate mixture of support material and the promoter source is produced,
f) the intimate mixture of support material and the promoter source is treated thermally, with the thermal treatment comprising a calcination process at a temperature in the range 200-1000° C., preferably 200-900° C. and particularly preferably 200-850° C.

The calcination process comprises preferably heating the catalyst at at least two different temperature levels.

The catalyst is preferably subjected to an activation treatment at 350 to 450° C. under 15 to 25 vol % H2 in inert gas for 1 to 30 h

The catalyst is preferably subjected to a conditioning treatment at 200 to 300° C. under the flow of 0.5 to 5 vol % ethanol for 12 to 48 h.

The catalyst is preferably subjected to an equilibration treatment under the conditions specified in any of claims 1 to 7 over 50 to 200 h time on stream.

The catalyst for condensing alcohols according to the particularly preferred embodiments is advantageously used in the process for condensing alcohols according to claims 9 to 15.

### Process for Condensing Alcohols

The invention also relates to a process for condensing alcohols by bringing an alcohol-comprising feed (gas) stream, where the alcohol is selected from the group consisting of C₁-C₅-alcohols, into contact with a catalyst in one of the forms presented here.

In the process of the invention for condensing alcohols using the catalyst of the invention,
(i) the process temperature is in the range from 200 to 450° C., preferably 250° C.-400° C.,
(ii) the process pressure is in the range 0.05-60 bar, more preferably 0.1-40 bar, particularly preferably 0.5-10 bar, even more preferably in the range 1-5 bar,
(iii) the alcohol content of the feed (gas) stream is in the range 0.1-90% by volume, preferably in the range 0.5-70% by volume and more preferably 0.5-50% by volume,
(iv) the feed (gas) stream has a GHSV in the range 500-5000 h⁻¹, preferably in the range 1000 4000 h-1, particularly preferably in the range 1000-2500 h⁻¹.

In a preferred embodiment of the process, the alcohol in the feed stream is ethanol which is converted into butanol.

The feed stream comprises alcohol and carrier fluid stream. The carrier fluid stream comprises inert gas and preferably inert gas together with reactive gas, with the reactive gas preferably being hydrogen. The inert gas is preferably nitrogen which itself does not undergo any reaction under the process conditions of the process of the invention. Possible inert gases are all gases which themselves do not undergo any reaction under the process conditions; in the industrial sector, the costs of the inert gases play a role, which would probably make the use of argon uneconomical.

In a preferred embodiment of the process of the invention, the process is carried out using a preferred ratio of alcohol to reactive gas since conversions, yields and selectivities can be improved further in this way. Particular preference is given to a ratio of alcohol to reactive gas in the range from 40:2.5 to 20:10, preferably from 20:2.5 to 20:20. In a further embodiment of the process, it is preferable for the ratio of alcohol to reactive gas to be in a range from 100:1 to 1:10.

In said embodiment, the process is preferably carried out using ethanol as alcohol and hydrogen as reactive gas.

The high performance of the process of the invention in respect of the high conversion, the good yields and selectivities to the target compound, preferably butanol, contribute, in particular, to an economical process having a high efficiency.

With regard to yields and selectivities, it should also be mentioned that the formation of by-products is not ruled out in the process of the invention for condensing alcohols. The by-products are formed in a much lower proportion than the main product or products. In the case of the by-products, a distinction has to be made between desirable by-products and undesirable by-products.

Products which are desirable in the reaction of ethanol are those products which can easily be converted into 1-butanol (1-BuOH), e.g. 1-butanal and crotonaldehyde. In addition, products which can be converted into products of value by after-treatment, e.g. acetaldehyde, also count as desirable by-products. Undesirable products are, in particular, gases such as CO, propane and methane which cannot be converted further under the present reaction conditions. In addition, high-boiling compounds without functional groups, which can be formed by uncontrolled further reaction of dimerization products, are undesirable.

If, in the condensation of ethanol, other compounds besides 1-BuOH are also formed, for example 1 butanal and crotonaldehyde, these may easily be converted to 1 BuOH in a subsequent hydrogenation step, for example.

In a preferred embodiment of the process of the invention for condensing alcohols, the process of the invention is preceded by a step in which the catalyst is thermally pretreated in a gas stream in order to activate and/or condition it. For example, the promoter elements can be converted into metallic species in the presence of a reducing atmosphere.

### Process for Condensing Different Alcoholic Components

A preferred embodiment of the process of the invention relates to the condensation of different alcoholic components. Processes for condensing alcohols from feed gas streams which comprise a plurality of different alcoholic components.

In this embodiment of the process for condensing alcohols in conjunction with the catalyst of the invention,
(i) the process temperature is implemented in the range from 200 to 450° C., preferably 250° C. to 400° C.,
(ii) the process pressure is in the range 0.05 to 60 bar, more preferably 0.1 to 40 bar, particularly preferably 5 to 9 bar,
(iii) the alcohol content of the feed (gas) stream is in the range 0.5 to 90% by volume, preferably in the range 0.5 to 70% by volume and more preferably in the range 0.5 to 50% by volume,
(iv) the feed (gas) stream has a GHSV in the range 500-5000 h⁻¹, preferably in the range 1000-4000 h⁻¹.

The GHSV (gas hourly space velocity) is the quotient of gas volume flow and catalyst volume.

In a further and preferred embodiment of the process of the invention, the alcohol-containing feed gas stream comprises at least two different alcoholic components from the group consisting of the C₁-C₅alcohols. The first of the at least two components is methanol (i.e. component 1), and the second of the at least two components (i.e. component 2) is a component from the group consisting of C₂-C₅alcohols. The second of the at least two components is preferably a component from the group consisting of C₂-C₄alcohols. Particular preference is given to the second of the at least two components being a C₂alcohol or a C₃alcohol.

Accordingly the present invention relates to a process for condensing alcohols by bringing an alcohol-comprising feed gas stream comprising an alcoholic component selected from the group consisting of C₁-C₅alcohols, especially at least two different alcoholic components from the group consisting of C₁-C₅alcohols, wherein very especially the first of the at least two components is methanol (i.e. component 1), and the second of the at least two components (i.e. component 2) is a component selected from the group consisting of C₂-C₅alcohols, into contact with the catalyst according to any of claims 1 to 8, wherein
(i) the process temperature is in the range from 200 to 450° C., preferably 250° C. to 400° C.,
(ii) the process pressure is in the range 0.05 to 60 bar, more preferably 0.1 to 40 bar, particularly preferably 5 to 9 bar,
(iii) the alcohol content of the feed (gas) stream is in the range 0.5 to 90% by volume, preferably in the range 0.5 to 70% by volume and more preferably in the range 0.5 to 50% by volume, and
(iv) the feed (gas) stream has a GHSV in the range 500-5000 h⁻¹, preferably in the range 1000-4000 h⁻¹.

When the process of the invention is carried out using the at least two different components, the molar proportion of methanol is preferably higher than the molar proportion of the at least second component from the group consisting of C₂-C₅alcohols. The molar ratio of methanol to component 2 (i.e. n methanol/n comp. 2) is preferably in the range from 5:1 to 50:1, more preferably in the range from 10:1 to 40:1 and in particular in the range from 15:1 to 20:1.

The C₁-C₅alcohols used in the process of the present invention can alternatively be renewable raw materials. For instance, bioethanol or alcohols derived from fusel oil may be used.

“Renewably-based” or “renewable” denote that the carbon content of a biofuel precursor and subsequent products is from a “new carbon” source as measured by ASTM test method D 6866-05, “Determining the Biobased Content of Natural Range Materials Using Radiocarbon and Isotope Ratio Mass Spectrometry Analysis”, incorporated herein by reference in its entirety. This test method measures the ¹⁴C/¹²C isotope ratio in a sample and compares it to the ¹⁴C/¹²C isotope ratio in a standard 100% biobased material to give percent biobased content of the sample.

Fusel oil is well known in the art and comprises a mixture of light alcohols, fatty esters, terpenes and furfural. The alcohols comprised in fusel oil are mainly propanol, butanol, amyl alcohol, isoamyl alcohols and hexanol and optionally heavier linear alcohols such as C₇ or C₈ alcohols.

Fusel oils, occasionally referred to as “amyl oils” or “fusels”, have compositions which vary depending on their origin (potato, beet, wheat, barley, etc. musts).

Fusel oil is a mixture of 5% to 20% of water, 60% to 95% of alcohols mainly consisting of linear or branched alkanols containing from 2 to 5 carbon atoms of impurities including but not limiting to furfurals, ethers, fatty acids, etc. which, may be up to 15%.

In an embodiment the composition of fusel oil is as follows:
Ethanol 5 to 40%,
1-Propanol 1 to 8%,
2-Propanol 0 to 1%,
2-Methylpropanol 5 to 15%,
1-Butanol-0 to 1%,
2-Methyl 1-butanol 10 to 30%,
   3-Methyl 1-butanol (isoamyl alcohol) 25 to 70%, the combination of alkanols representing 100%.

In addition to the alcoholic components, the feed gas stream comprises a carrier gas. The carrier gas is inert gas (for example nitrogen) or reactive gas (for example hydrogen). In a preferred embodiment, the feed gas comprises reactive gas, with the reactive gas being able to be present together with inert gas.

In a further embodiment, the process of the invention relates to the condensation of methanol and n-propanol to produce iso-butanol. The proportion of methanol in the feed gas stream is preferably from five to ten times higher than the proportion of n-propanol.

In a particularly preferred embodiment, the process of the invention relates to the condensation of methanol and ethanol to produce iso-butanol. The embodiment relates to a process in which one ethanol molecule is reacted with two methanol molecules. The proportion of methanol in the feed gas stream is preferably from five to twenty times, more preferably from fifteen to twenty times higher than the proportion of ethanol.

In the various embodiments of the process of the invention, it is important that the feed gas stream is brought into contact under the reaction condition specified in the disclosure with the catalyst of the invention, which is disclosed in more detail in the present description and the claims, and the alcoholic components are reacted.

As regards the ratio of alcohol-containing components to reactive gas, preference is given to this being in the range from 40:2.5 to 20:10, preferably from 20:2.5 to 20:20. Further preference is given to an embodiment of the process of the invention in which hydrogen is used as reactive gas.

In a further embodiment of the process, the ratio of alcohol to reactive gas is preferably in the range from 100:1 to 1:10. It is known to those skilled in the art that the amount of reactive gas used also depends on the respective process parameters and the information given here is not intended to constitute restrictions. In particular, optimization of the process parameters can also lead to the preferred amount of reactive gas used depending on the respective alcohol components and the reaction conditions.

On the reaction of EtOH and MeOH a reaction temperature of 325° C. using a feed comprising the EtOH and MeOH in a molar ratio of 1:20 in the presence of 20% by volume of H₂ at GHSV 1000 h⁻¹ at 7 bar reaction pressure results in high conversion, the good yields and selectivities to the target compound, preferably iso-butanol. In said embodiment the proportion of copper promoter in the catalyst is in the range 0.05 to 10% by weight, especially 0.05 to 5.0% by weight, such as, for example, 0.1 or 1.0% by weight and no further promoter element is present.

When copper catalysts are equilibrated under mixed Guerbet conditions (temperature of 325° C. using a feed comprising the EtOH and MeOH in a molar ratio of 1:20 in the presence of 20% by volume of H₂ at 7 bar reaction pressure) over 100 h time on stream, the selectivity of Cu catalysts can be changed from CO/CO₂ to Guerbet intermediates such as C₃/C₄ aldehydes and C₃/C₄ alcohols. This equilibration phase may also lead to the complete disappearance of CO₂ from the product spectrum over time on stream.

For example, a catalyst containing 0.1% by weight copper was more selective to C₄/C₃ products at the beginning and a catalyst containing 1.0% by weight copper produced more CO/CO₂, as with time on stream, the catalyst containing 1.0% by weight copper showed improved selectivity almost the same as the catalyst containing 0.1% by weight copper and also good conversion of EtOH and MeOH.

The high performance of the process of the invention in respect of the high conversion, the good yields and selectivities to the target compound, preferably iso-butanol, contribute, in particular, to an economical process having a high efficiency.

With regard to yields and selectivities, it should also be mentioned that the formation of by-products is not ruled out in the process of the invention for condensing alcohols. The by-products are formed in a much lower proportion than the main product or products. In the case of the by-products, a distinction has to be made between desirable by-products and undesirable by-products.

Products which are desirable in the reaction of ethanol are those products which can easily be converted into isobutanol, e.g. isobutanal and crotonaldehyde. In addition, products which can be converted into products of value by after-treatment, e.g. acetaldehyde, also count as desirable by-products. Undesirable products are, in particular, gases such as CO, propane and methane which cannot be converted further under the present reaction conditions. In addition, high-boiling compounds without functional groups, which can be formed by uncontrolled further reaction of dimerization products, are undesirable. The obtained isobutanol is separated and purified by methods, such as, for example, distillation.

In a further preferred embodiment, after the process of the invention has been carried out, the catalyst is treated by a regeneration process, which contributes to the catalyst regaining at least a large proportion of its initial activity.

With regard to the catalyst of the invention for condensing alcohols, it may be said that the catalyst precursor material is preferably free of ethylenediaminetetraacetic acid or anions of ethylenediaminetetraacetic acid. Preference is additionally given to the intimate mixture being produced from support material comprising a hydrotalcite-like compound, preferably hydrotalcite-comprising support material, and/or precursor material of a hydrotalcite-like compound, preferably hydrotalcite precursor material, and the promoter source, where the mixture preferably does not comprise any ethylenediaminetetraacetic acid or anions of ethylenediaminetetraacetic acid.

Additional aspects of the present invention relate to the use of the isobutanol, obtained according to the process described above and/or obtained according to the process of any of claims 9 to 15, preferably from sources of renewable raw materials, as starting material in the synthesis of isoprenol (3-methylbut-3-en-1-ol), prenol (3-methyl-2-buten-1-ol), Citral (3,7-dimethylocta-2,6-dienal), Linalool (3,7-dimethyl-1,6-octadien-3-ol), Menthol (5-methyl-2-(propan-2-yl)-cyclohexan-1-ol) and Vitamin A ((2E,4E,6E,8E)-3,7-dimethyl-9-(2,6,6-trimethylcyclohex-1-enyl) nona-2,4,6,8-tetraen-1-ol).

In a first aspect, the invention provides a process for the preparation of isoprenol, comprising the steps of:
a-i) subjecting the isobutanol, obtained according to the process described above and/or obtained according to the process of any of claims 9 to 15, to dehydration so as to obtain isobutylene; and
a-ii) reacting at least one formaldehyde source and the isobutylene obtained in step a-i) to obtain isoprenol.

In a second aspect, the invention provides a process for the preparation of prenol, comprising the steps of:
a) providing isoprenol by a-i) and a-ii):
   a-i) subjecting the isobutanol, obtained according to the process described above and/or obtained according to the process of any of claims 9 to 15, to dehydration so as to obtain isobutylene;
   a-ii) reacting at least one formaldehyde source and the isobutylene obtained in step a-i) to obtain isoprenol; and
b) isomerizing isoprenol obtained in step a) to obtain prenol by bringing a reactant stream comprising isoprenol into contact with at least one heterogeneous isomerization catalyst, preferably in the presence of hydrogen.

In a third aspect, the invention provides a process for the preparation of prenal and/or isoprenal, comprising the steps of:
a) providing isoprenol by a-i) and a-ii):
   a-i) subjecting the isobutanol, obtained according to the process described above and/or obtained according to the process of any of claims 9 to 15, to dehydration so as to obtain isobutylene;
   a-ii) reacting at least one formaldehyde source and the isobutylene obtained in step a-i) to obtain isoprenol;
b) optionally, isomerizing isoprenol obtained in step a) to obtain prenol by bringing a reactant stream comprising isoprenol into contact with at least one heterogeneous isomerization catalyst, preferably in the presence of hydrogen; and
c) providing prenal by at least one of c-i) and c-ii):
   c-i) subjecting isoprenol obtained in step a) to oxidative dehydrogenation so as to obtain prenal and/or isoprenal by bringing a reactant stream comprising isoprenol into contact with at least one heterogeneous oxidative dehydrogenation catalyst, in the presence of molecular oxygen, and optionally isomerizing at least part of the isoprenal to prenal; and
   c-ii) oxidizing prenol obtained in step b) so as to obtain prenal by bringing a reactant stream comprising prenol into contact with at least one oxidant and at least one oxidation catalyst, preferably in the presence of a liquid phase.

In a fourth aspect, the invention provides a process for the preparation of 3,7-dimethyl-octa-2,6-dienal (citral) comprising the steps of:
a) providing isoprenol by a-i) and a-ii):
   a-i) subjecting the isobutanol, obtained according to the process described above and/or obtained according to the process of any of claims 9 to 15, to dehydration so as to obtain isobutylene;
   a-ii) reacting at least one formaldehyde source and the isobutylene obtained in step a-i) to obtain isoprenol;
b) isomerizing isoprenol obtained in step a) to obtain prenol by bringing a reactant stream comprising isoprenol into contact with at least one heterogeneous isomerization catalyst, preferably in the presence of hydrogen;
c) providing prenal by at least one of c-i) and c-ii):
   c-i) subjecting isoprenol obtained in step a) to oxidative dehydrogenation so as to obtain prenal and/or isoprenal by bringing a reactant stream comprising isoprenol into contact with at least one heterogeneous oxidative dehydrogenation catalyst, in the presence of molecular oxygen, and optionally isomerizing at least part of the isoprenal to prenal;
   c-ii) oxidizing prenol obtained in step b) so as to obtain prenal by bringing a reactant stream comprising prenol into contact with at least one oxidant and at least one oxidation catalyst, preferably in the presence of a liquid phase;
d) condensing prenol obtained in step b) with prenal obtained in step c) to obtain diprenyl acetal of prenal; and
e) subjecting diprenyl acetal of prenal (3-methyl-2-butenal-diprenylacetal) obtained in step d) to cleaving conditions to obtain citral.

### First Aspect—Preparation of Isoprenol

### Subjecting Isobutanol to Dehydration to Obtain Isobutylene

Isobutanol is subjected to dehydration in the presence of a catalyst so as to obtain isobutylene. Reference is made to Jean-Luc Dubois et al., Catalysis Today 418 (2023) 114126.

Isobutylene can, for example, be produced according to the method described in EP4129963A1, comprising producing isobutylene from isobutanol using the catalyst described therein.

The catalyst according to EP4129963A1 contains at least one metal selected from Group 6 to Group 14 metal elements in Period 4 to Period 6 of the periodic table, which is preferably selected from Mn, Fe, Co, Ni, Cu, and Zn, in alumina which includes alumina consisting of one or more crystal phases of a monoclinic crystal phase, a tetragonal crystal phase, and a cubic crystal phase, such as, for example, alumina containing γ-alumina having a tetragonal crystal phase as a main component, BET specific surface area: 243 m²/g, Na₂O content: less than 0.050% by mass, SiO₂ Content: 0.10% by mass. The content of the metal is preferably 0.025 mmol or more with respect to 1 g of the alumina.

Dehydration of isobutanol may be carried out in the liquid phase or in the gas phase. When the reaction is carried out in the gas phase, a type of a fixed bed, a fluidized bed, or the like can be used.

For example, by vaporizing a raw material with a vaporizer, it can be supplied to a reactor as a raw material gas. The conditions for vaporizing the raw material are not particularly limited, and for example, the temperature can be 108° C. or higher and 600° C. or lower, and the pressure can be 0.05 MPa or higher and 1 MPa or lower in terms of absolute pressure. In the raw material gas, the isobutanol concentration can be adjusted by diluting isobutanol with a diluent gas. The raw material gas may be a gas consisting only of isobutanol. The diluent gas may be any gas that does not affect the dehydration of isobutanol. Oxygen or hydrogen may be used as a diluent gas. The diluent gas included in the raw material gas may be a mixture of two or more diluent gases. Moisture may be included in the raw material gas. The isobutanol concentration in the raw material gas is preferably 5.0% by volume or more, particularly preferably 15.0% by volume or more, even still more preferably 25.0% by volume or more, and most preferably 45.0% by volume or more, with respect to the total volume of the raw material gas. The upper limit is not particularly limited, and it is 100% by volume or less. The reaction temperature in the dehydration of isobutanol is preferably 200° C. or higher, more preferably 220° C. or higher, still more preferably 240° C. or higher, particularly preferably 260° C. or higher, and most preferably 280° C. or higher. The reaction pressure in the dehydration of isobutanol is preferably 50 kPa or more in terms of absolute pressure and preferably 600 kPa or less.

Alternatively, the method for producing isobutylene described in CN106582603A may be used, which comprises subjecting isobutanol to a dehydration reaction in the presence of a modified alumina catalyst produced by a process comprising the steps of:
a) mixing an aluminum salt solution and an alkali liquor;
b) aging the mixture obtained in the step a) to obtain pseudo-boehmite gel;
c) mixing the pseudo-boehmite gel obtained in the step b) with amorphous silica-alumina, drying, extruding and molding to obtain a carrier;
d) impregnating the carrier obtained in step c) with caustic alkali solution and calcium salt solution, drying and roasting.

Alternatively, the method described in WO2022/226371 may be used, which is directed to a process for converting one or more C₁-C₅ linear or branched alcohols to one or more C₂-C₅ olefins, the process comprising: contacting an input stream comprising the one or more C₁-C₅ linear or branched alcohols with at least a first catalyst and a second catalyst in a single bed reactor to form an output stream comprising the one or more C₂-C₅ olefins, the single bed reactor being at a temperature from about 350° C. to about 750° C., a gauge pressure from 0 to about 30 bar, and a weight hourly space velocity (WHSV) from about 0.5 to about 5.0, wherein the first catalyst comprises a doped or undoped alumina catalyst including, in neutral or ionic form, one or more of zirconium (Zr), titanium (Ti), tungsten (W), or silicon (Si), to form a first mixture; and wherein the second catalyst comprises a doped or undoped zeolite catalyst.

Alternatively, the method for producing isobutylene described in U.S. Pat. No. 10,464,860 may be used, which comprises introducing a reaction gas containing isobutanol into a reactor, wherein the reaction gas further contains water in a content of 0.1 to 70 vol %; and dehydrating the isobutanol at a reaction pressure of 50 kPa or more and 750 kPa or less as an absolute pressure by using an alumina catalyst having a BET specific surface area in a range of 60 m²/g or more and 175 m²/g or less and having a content of SiO₂ of less than 1.0 mass %.

In a preferred embodiment of U.S. Pat. No. 10,464,860 a reaction gas containing isobutanol is introduced into a reactor, wherein the reaction gas further contains water in a content of 0.1 to 70 vol %; and the isobutanol is dehydrated at a concentration of isobutanol relative to all gaseous components entering a reaction zone to be supplied of 30 vol % or more and 85 vol % or less, a weight hourly space velocity (WHSV) of isobutanol of 0.175 h⁻¹ or more and 20 h⁻¹ or less, and a reaction pressure of 50 kPa or more and 750 kPa or less as an absolute pressure, by using an alumina catalyst of which 90 mass % or more has a particle size in a range of 700 μm or more and 10000 μm or less.

The isobutylene may be separated and purified from a reaction gas containing isobutylene and unreacted isobutanol by the method described, for example, in U.S. Pat. No. 10,550,052B2 comprising: a step (1) of contacting the reaction gas containing the isobutylene and unreacted isobutanol with a first solvent to obtain a first gas containing the isobutylene and a recovered solution containing the unreacted isobutanol;
a step (2) of contacting the first gas with a second solvent selected from tert-butanol, a tert-butanol aqueous solution, and methyl tert-butyl ether to allow the second solvent to absorb the isobutylene contained in the first gas to obtain an absorption solution containing the isobutylene, and a step (3) of distilling the absorption solution to obtain separated and purified isobutylene.

### Reacting a Formaldehyde Source and Isobutylene to Obtain Isoprenol

The isobutylene obtained in step a-i) is reacted in step a-ii) with at least one formaldehyde source to obtain 3-methylbut-3-en-1-ol (isoprenol). The at least one formaldehyde source and isobutylene are typically reacted in a reactor, in general at elevated temperature and pressure.

As used herein, “formaldehyde source” refers to any source containing formaldehyde or capable of cleaving off formaldehyde. Formaldehyde sources include aqueous formaldehyde solutions and oligomers or polymers of formaldehyde, like paraformaldehyde.

In one embodiment, the isoprenol is obtained by introducing, preferably by mixing and injecting, at least one formaldehyde source and isobutylene into a reactor, preferably through at least one nozzle, and reacting the at least one formaldehyde source and isobutylene under supercritical conditions. In order to achieve supercritical conditions, formaldehyde and isobutylene are preferably reacted at a temperature of at least 220° C., for example in the range of 220 to 290° C., and an absolute pressure of at least 200 bara. The reaction of isobutene and formaldehyde may be carried out without a catalyst as well as in the presence of at least one catalyst. The reaction of isobutylene and formaldehyde source may also be carried out in the presence of one or more auxiliary chemicals such as ammonia and/or hexamethylenetetramine (urotropin). Conducting this reaction in the presence of such auxiliary chemicals, especially ammonia and/or urotropin, has been described, e.g., in DE1279014B.

The at least one formaldehyde source and isobutylene are preferably introduced into the reactor in a manner which allows for mixing of the reactants so as to obtain an intimate mixture. Introduction methods include injecting, splashing, stirring in and/or spraying into the reactor. Preferably, the at least one formaldehyde source and isobutylene are injected or sprayed into the reactor through at least one nozzle.

Formaldehyde may be provided as a liquid, for example as a solution of paraformaldehyde in methanol. Preferably, the at least one formaldehyde source comprises or is an aqueous formaldehyde solution.

While initial rapid and intense mixing of reactants is desirable, it may be advantageous to continue and complete the reaction under conditions of limited back-mixing. Thus, the reaction mixture may be passed into a post-reaction chamber disposed after the reactor or in a lower portion of the reactor. In the post-reaction chamber, back-mixing is limited.

In one embodiment, the reactor comprises an upper portion and a lower portion. Introduction of the reactants, in particular by injecting and mixing of the reactants, occurs in a mixing chamber of the reactor disposed in the upper portion, and a fluid comprising formaldehyde and/or isobutylene and/or isoprenol is passed from the mixing chamber into a post-reaction chamber disposed in the lower portion.

In one embodiment, reacting at least one formaldehyde source and isobutylene comprises introducing, preferably mixing and injecting, the at least one formaldehyde source and isobutylene into an internal loop reactor through at least one nozzle into first conduit(s), the internal loop reactor comprising:
a vertically disposed cylindrical vessel comprising a sidewall;
at least one draft tube having a tube inlet end and a tube outlet end, arranged vertically within the vessel, the draft tube(s) being arranged concentrically to the nozzle(s), and having an inner surface and an outer surface, wherein the draft tube(s) provide(s) the first conduit(s) within the draft tube(s), and a second conduit outside of the draft tube(s) and within the sidewall, the first conduit(s) being in fluid communication with the second conduit;
reactor fluid outlet means;
   wherein the inner surface of the draft tube(s) convexly curves so that the first conduit(s) exhibit(s) an annular constriction of the cross-section between the tube inlet end and the tube outlet end; wherein the constriction is located closer to the tube inlet end; wherein the convex curvature of the inner surface of the draft tube(s) extends over at least 70%, preferably at least 80%, most preferably at least 90% of the length of the draft tube; and
   wherein the outer surface of the draft tube(s) convexly curves so that the draft tube(s) exhibit(s) a circumferential protuberance between the tube inlet end and the tube outlet end, which circumferential protuberance is preferably located closer to the tube outlet end; wherein the convex curvature of the outer surface of the draft tube(s) extends over at least 70%, preferably at least 80%, most preferably at least 90% of the length of the draft tube; and
   wherein the edges of the draft tube(s) are rounded so that the at least one formaldehyde source and isobutylene introduced through the nozzles travel generally downward in the first conduit(s) to obtain a reacted fluid, the reacted fluid is then diverted in the opposite direction so as to travel through the second conduit and is subsequently back-mixed with the introduced fluid.

In a preferred embodiment, the nozzles are two-component nozzles. It is especially preferable that a two-component nozzle is designed so as to provide an annular jet of isobutylene around a central jet of the at least one formaldehyde source, and that the velocities upon introduction, for example the injection velocities or spraying velocities, of these two jets are different. In this embodiment, the jet of isobutylene has a large shear surface towards both the central jet of the at least one formaldehyde source and the reaction mixture in the reactor, allowing for favorable fast mixing of the reactants.

In a preferred embodiment, the loop reactor comprises deflector means arranged between the nozzle and the draft tube, the deflector means being suitable for deflecting fluid travelling in the second conduit in the opposite direction.

The deflector means suitably comprise a surface which is concave relative to the end of the draft tube which defines the tube inlet end. In a preferred embodiment, the deflector means have a partial toroidal surface. It is especially preferred that the deflector means are provided in the shape of the upper portion of a ring torus bisected in a plane parallel to the toroidal direction. This shape allows for an especially efficient deflection of the fluid travelling in the second conduit. The deflector means may allow for a stabilization of the introduced, for example injected or sprayed fluid stream. This is especially relevant when the flow rate of the fluid travelling in the second conduit is not uniform across the cross section of the reactor, which may lead to an eccentricity of the introduced fluid stream. Such an eccentricity may cause a decrease in circulation ratio if left unattended.

When the first conduit is downcomer conduit and the second conduit is a riser conduit, it is preferred that the shape of the deflector means constitutes the upper portion of a ring torus bisected in a plane parallel to the toroidal direction, wherein the ring torus is bisected at least 50% of its height, such as at least 55% or 65% of its height. Thus, the upper portion of the ring torus is the same size or smaller than the lower portion of the ring torus. In another preferred embodiment, the shape of the deflector means constitutes the upper portion of a ring torus bisected in a plane parallel to the toroidal direction wherein the ring torus is bisected at at most 85% of its height, for example 80% of its height. In these ranges, the entry of the deflector means is angled especially suitable for fluid deflection.

Further details regarding aforementioned embodiments concerning loop reactor may be found in WO2023/104863, which herewith is incorporated by reference in its entirety.

High temperatures are required to obtain a high isoprenol yield in the reaction of formaldehyde with isobutylene. Effective removal of the heat is critical for the product quality and process safety. The heat removed from the isoprenol is used for raising the temperature of isobutylene before it enters the reactor. The stream of the hot isoprenol contains sensible heat from the chemical reaction. The sensible heat is potentially reclaimable energy that can be reused.

Advantageously, reacting at least one formaldehyde source and isobutylene preferably comprises heat-exchanging a stream of hot isoprenol withdrawn from the reactor with a isobutylene stream directed to the reactor; wherein heat-exchanging is performed in one or more shell-and-tube heat exchangers; each of the heat exchangers comprising a plurality of tubes and a shell-side heat exchange passage; wherein the hot isoprenol is directed through the tubes of the heat exchangers; and the isobutylene is guided through the shell-side passage, and in case of more than one heat exchangers at least two of the heat exchangers are connected in series with regard to both the shell-side flow and the tube-side flow.

In a group of the preferred embodiments, the heat-exchanging is performed in one shell-and-tube heat exchanger.

In another group of the preferred embodiments, the heat-exchanging is performed in at least one or more shell-and-tube heat exchangers, wherein the hot isoprenol is directed through the tubes of the heat exchangers; and the isobutylene is guided through the shell-side passage, and in case of at least two of the heat exchangers these are connected in series with regard to both the shell-side flow and the tube-side flow.

Such configurations allow for prolonging operation intervals between maintenance disruptions in such a method. The term “maintenance disruptions” is intended to mean a shutdown of the process that becomes necessary at recurring intervals in order to clear the tubes of the heat exchanger that have been clogged by fouling. An indicator of a necessity of a maintenance disruption is typically when isobutylene leaving the last heat exchanger is insufficiently pre-heated and that even a subsequent heater is hardly able to put in additional external heat into the isobutylene to bring isobutylene to the required temperature before it enters the reactor. One aspect of the invention is that the pre-heating of the isobutylene stream can be maintained for a longer time at levels high enough so that the desired temperature of the isobutylene can easily be reached before the isobutylene enters the reactor.

One particular area prone to fouling in conventional shell-and-tube heat exchangers is the tube area near the tube sheet near the inlet where the tube-side fluid leaves the individual tubes. Excessive fouling in this area can cause clogging of individual tubes and fluid stagnation along the entire length of these tubes. The fluid stagnation generally leads to reduced heat-transfer performance.

As a further consequence of the decreased heat transfer performance caused by fouling, the energy required in a heater to adjust the temperature of the pre-heated isobutylene stream to the desired reaction temperature increases. Consequently, more additional external heat becomes necessary which is detrimental in terms of energy demand and process economy, and often has a negative impact on the carbon dioxide footprint of the product.

By using two or more heat exchangers, the impact of fouling in individual tubes on the overall heat exchange capacity is reduced in comparison to arrangements where only a single heat exchanger is used. As a consequence, the heat transfer rates are maintained at a desired level for longer periods, hence prolonging operation intervals between maintenance disruptions, and the pre-heating of the isobutylene stream requires less additional external heat compared to a plant with a single heat exchanger in an advanced state of fouling.

Further details regarding aforementioned embodiments concerning heat exchangers and energy savings and reducing maintenance intervals may be found in WO 2023/198714 A1, which herewith is incorporated by reference in its entirety.

### Second Aspect—Preparation of Prenol

The second aspect of the invention relates to the preparation of 3-methyl-2-buten-1-ol (prenol), comprising a) providing isoprenol as described above via steps a-i) and a-ii) according to the first aspect, and b) isomerizing isoprenol obtained in step a) to obtain prenol by bringing a reactant stream comprising isoprenol into contact with at least one heterogeneous isomerization catalyst, preferably in the presence of hydrogen.

As used herein and hereinafter, the term “reactant stream” refers to a stream comprising a reactant or reactants consumed in the course of a chemical reaction. In this sense, the reactant stream may further comprise solvent(s), catalyst(s), additive(s) and/or any other substance involved in the chemical reaction.

Generally, the isomerization of isoprenol to 3-methyl-2-buten-1-ol (prenol) may be carried out over a supported noble metal, preferably in the presence of hydrogen.

A preferred catalyst is a fixed bed catalyst containing palladium and selenium or tellurium or a mixture of selenium and tellurium supported on silicon dioxide.

The catalyst contains 0.1 to 2.0% by weight of palladium and 0.01 to 0.2% by weight of selenium, tellurium or a mixture of selenium and tellurium, based on the total weight of the catalyst.

The BET surface area is, for example, in the range of 100 to 150 m²/g, in particular in the range of 110 to 130 m²/g. The BET surface area is determined by N₂ adsorption according to DIN 66131.

The pore volume in the pore diameter range from 3 nm to 300 μm is preferably 0.8 to 0.9 cm³/g, in particular 0.8 to 0.85 cm³/g. Thereby, 80 to 95%, preferably 85 to 93% of this pore volume is in the pore diameter range of 10 to 100 nm. The pore volume is determined by Hg porosimetry.

Preferably, the catalyst contains 0.2 to 0.8% by weight, in particular 0.4 to 0.6% by weight of palladium. Preferably, the catalyst contains 0.02 to 0.08, in particular 0.04 to 0.06 wt % selenium, tellurium or a mixture of selenium and tellurium, preferably selenium. In addition to the active components mentioned, other metals may be present on the catalyst in small amounts. Preferably, only palladium, selenium and/or tellurium, in particular only palladium and selenium, are present on the silica support.

The described isomerization of isoprenol to prenol on a fixed-bed catalyst is also described in EP-A-841090, to which express reference is made.

The isomerization is carried out at a temperature in the range of 50 to 150° C., preferably in the range of 60 to 130° C., more preferably in the range of 70 to 120° C. to produce a reaction mixture of prenol and isoprenol. The isoprenol can be recycled. Further details are provided in WO2008/037693.

Generally, a regeneration cycle is performed periodically, to remove accumulated coke from the catalyst. The regeneration cycle can be initiated when the pressure drop increased above a threshold value, or at arbitrary time intervals, for example once a week. A regeneration cycle consists of sending diluted air or air for a defined period of time, for example 6 to 24 h, over the reactor while increasing the salt bath temperature, for example 400 to 450° C., to allow coke combustion.

The unreacted isoprenol from the isoprenol isomerization process may be used, i.e. recycled for the isoprenol isomerization.

### Reducing the Content of Aldehydes in the Reactant Stream

The presence of aldehydes, especially formaldehyde and/or prenal in the reactant stream, is detrimental to the activity and selectivity of the process and may accelerate catalyst deactivation and/or poisoning in the isomerization of isoprenol to prenol.

As used herein and hereinafter, the term “concentration of aldehydes in the reactant stream” refers to the total concentration of aldehydes existing in the reactant stream. Aldehydes include those intrinsic to the isoprenol preparation process and those formed by oxidation and isomerization. Hence, the aldehydes usually include formaldehyde and prenal. Therefore, if formaldehyde and prenal are the only aldehydes existing in the reactant stream, the concentration of aldehydes in the reactant stream is the sum of the respective concentrations of formaldehyde and prenal.

The deterioration of catalyst properties is related to the presence of aldehydes, especially formaldehyde and/or prenal in the reactant stream. Formaldehyde is generally considered to be the most critical of these aldehydes. Catalyst-fouling reactions of condensation and polymerization are believed to be the principal reactions involved in carbon or coke formation on the catalyst. It is thought that this carbon formation involves thermal condensation of aldehydes, for example formaldehyde and/or prenal, or of these aldehydes with the olefinic hydrocarbons isoprenol. In the presence of the catalyst the primary condensation products tend to undergo dehydrogenation and polymerization type reactions and to settle on the catalyst and undergo further dehydrogenation and decomposition until carbonaceous deposits are formed.

One of the poisoning mechanisms of the catalyst is supposed to involve a catalytic or non-catalytic dehydrogenation of aldehydes, especially formaldehyde and/or prenal to carbon monoxide, which is chemisorbed on the catalyst and blocks the active centers.

A further cause of catalyst deactivation, which may occur in combination with the previously mentioned cause of catalyst poisoning, is the formation of paraformaldehyde or trioxane which may deposit, in the form of solids, on the catalyst and shield the catalytically active surfaces from the isoprenol being processed. This leads to progressive deactivation of the catalyst.

In one embodiment, the concentration of aldehydes in the reactant stream is, therefore, maintained at a certain level or less, i.e. less than 0.5% by weight, preferably less than 0.4% by weight, in particular less than 0.3% by weight, or less than 0.25% by weight, based on the total weight of the reactant stream, wherein the concentration of aldehydes in the reactant stream is not lower than 10 ppm, preferably not lower than 25 ppm, in particular not lower than 50 ppm, or not lower than 100 ppm, with respect to the total weight of the reactant stream. As used herein and hereinafter, the term “ppm” refers to parts-per-million (ppm, 10-6).

In more preferred embodiments, the concentration of aldehydes is maintained at less than 0.2% by weight, based on the total weight of the reactant stream, wherein the concentration of aldehydes in the reactant stream is not lower than 10 ppm, preferably not lower than 25 ppm, in particular not lower than 50 ppm, or not lower than 100 ppm, with respect to the total weight of the reactant stream.

Aldehydes, preferably formaldehyde and/or prenal may be removed from the streams comprising isoprenol by a conventional separating method such as distillation, selective adsorption and or selective reaction, in particular by the purification process involving the pressure-swing distillation as described herein.

Alternatively, it is feasible to mix the unreacted isoprenol stream with an amount of a sufficiently purified fresh feed stream so as to give in the combined stream a desired weight ratio of formaldehyde to isoprenol.

As used herein and hereinafter, the term “unreacted isoprenol stream” refers to a stream which is derived from an isoprenol isomerization process and comprises unreacted isoprenol of the isoprenol isomerization process. In this sense, the unreacted isoprenol stream may further comprise solvent(s), catalyst(s), additive(s) and/or any other substance involved in the isoprenol isomerization process.

As used herein and hereinafter, the term “crude isoprenol stream” refers to a product stream of an isoprenol production process from which unreacted isobutylene has been removed.

Removal of aldehydes, such as formaldehyde and/or prenal, is accomplished in a purification unit following the isoprenol synthesis. A preferred method of recovering aldehydes from a crude isoprenol stream to which an unreacted isoprenol stream is admixed, is described in more detail below.

Preferably, the aldehydes existing in the reactant stream comprise formaldehyde. Also preferably, the aldehydes existing in the reactant stream comprise prenal besides formaldehyde.

More preferably, the aldehydes existing in the reactant stream consist of prenal and formaldehyde. In certain instances, the aldehydes existing in the reactant stream consist of formaldehyde.

Preferably, the concentration of aldehydes in the reactant stream is less than 0.5% by weight, or 0.4% by weight, or 0.3% by weight, more preferably less than 0.25% by weight, or 0.2% by weight, even more preferably less than 0.15% by weight, yet even more preferably less than 0.1% by weight, equal to or less than 0.08% by weight or less than 0.05% by weight, based on the total weight of the reactant stream, but at least 10 ppm with respect to the total weight of the reactant stream. In another embodiment, the concentration of aldehydes is less than 0.025% by weight, more less than 0.02% by weight, based on the total weight of the reactant stream. In one embodiment, the concentration of aldehydes in the reactant stream is not lower than 10 ppm, preferably not lower than 25 ppm, in particular not lower than 50 ppm, or not lower than 100 ppm, with respect to the total weight of the reactant stream. The skilled person will appreciate that any of the upper limits of aldehyde concentration can be combined with any of the lower limits of aldehyde concentration, wherein in certain embodiments the aldehyde is either formaldehyde, prenal, or formaldehyde and prenal.

Also preferably, the concentration of formaldehyde in the reactant stream is less than 0.5% by weight, or 0.4% by weight, or 0.3% by weight, more preferably less than 0.25% by weight, or 0.2% by weight, even more preferably less than 0.15% by weight, yet even more preferably less than 0.1% by weight, equal to or less than 0.08% by weight or less than 0.05% by weight, based on the total weight of the reactant stream, but at least 10 ppm with respect to the total weight of the reactant stream. In another embodiment, the concentration of formaldehyde is less than 0.025% by weight, more less than 0.02% by weight, based on the total weight of the reactant stream, wherein the concentration of formaldehyde in the reactant stream is not lower than 10 ppm, preferably not lower than 25 ppm, in particular not lower than 50 ppm, or not lower than 100 ppm, with respect to the total weight of the reactant stream.

If the aldehydes existing in the reactant stream comprise or consist of formaldehyde, the concentration of formaldehyde in the reactant stream is preferably less than 0.5% by weight, or less than 0.4% by weight, or less than 0.3% by weight, more preferably less than 0.25% by weight, or 0.2% by weight, even more preferably less than 0.15% by weight, yet even more preferably less than 0.1% by weight, or less than 0.05% by weight, most preferably less than 0.025% by weight, or less than 0.02% by weight, based on the total weight of the reactant stream, wherein the concentration of aldehydes in the reactant stream is not lower than 10 ppm, preferably not lower than 25 ppm, in particular not lower than 50 ppm, or not lower than 100 ppm, with respect to the total weight of the reactant stream.

Preferably, the concentration of prenal in the reactant stream is less than 0.3% by weight, more preferably less than 0.2% by weight, even more preferably less than 0.15% by weight, in particular less than 0.1% by weight, based on the total weight of the reactant stream, but not lower than 10 ppm, preferably not lower than 25 ppm, in particular not lower than 50 ppm, or not lower than 100 ppm, with respect to the total weight of the reactant stream.

Therefore, in preferred embodiments, the aldehydes in the reactant stream consist of or comprises formaldehyde, and the concentration of formaldehyde is less than 0.5% by weight, or less than 0.4% by weight, or less than 0.3% by weight, more preferably less than 0.25% by weight, or 0.2% by weight, even more preferably less than 0.15% by weight, yet even more preferably less than 0.1% by weight, equal to or less than 0.08% by weight, or less than 0.05% by weight, based on the total weight of the reactant stream, but at least 10 ppm with respect to the total weight of the reactant stream. In another embodiment, the concentration of formaldehyde is less than 0.025% by weight, more preferably less than 0.02% by weight, based on the total weight of the reactant stream, but not less than 10 ppm, preferably not lower than 25 ppm, in particular not lower than 50 ppm, or not lower than 100 ppm, with respect to the total weight of the reactant stream. In one embodiment, the concentration of formaldehyde is equal to or less than 0.08% by weight, based on the total weight of the reactant stream, but optionally at least 10 ppm with respect to the total weight of the reactant stream.

In an embodiment, the aldehydes existing in the reactant stream consist of prenal and formaldehyde, and therefore the concentration of aldehydes in the reactant stream corresponds to the sum of the concentrations of prenal and formaldehyde, wherein the concentration of aldehydes in the reactant stream, i.e. the sum of the concentrations of prenal and formaldehyde is less than 0.5% by weight, preferably less than 0.4% by weight, in particular less than 0.3% by weight or less than 0.2% by weight, based on the total weight of the reactant stream, wherein the concentration of aldehydes in the reactant stream is not lower than 10 ppm, preferably not lower than 25 ppm, in particular not lower than 50 ppm, or not lower than 100 ppm, with respect to the total weight of the reactant stream.

In a group of the preferred embodiments, the weight ratio of aldehydes, preferably prenal and/or formaldehyde, to isoprenol in the reactant stream is adjusted at a certain level or less, i.e. less than 0.04, preferably less than 0.03, in particular less than 0.02, or less than 0.01. In still more preferred embodiments, the weight ratio of aldehydes, preferably formaldehyde and/or prenal to isoprenol is adjusted at less than 0.002, or less than 0.001. In one embodiment, the ratio is lower than 0.0009.

The terms “maintaining in the reactant stream” and “adjusting in the reactant stream” or “maintained in the reactant stream” or “adjusted in the reactant stream” with respect to the aldehyde levels in the reactant stream are used interchangeably herein.

Reducing the weight ratio of aldehydes, preferably formaldehyde and/or prenal to isoprenol in the reactant stream beyond a certain point, however, reaches a point of rapidly diminishing return. Removal of aldehydes, especially of formaldehyde and/or prenal involves additional equipment and operating costs. An economic balance must be taken between the improvement due to reducing the ratio and the cost of maintaining such a ratio.

Hence, the weight ratio of aldehydes, preferably formaldehyde and/or prenal to isoprenol is preferably not lower than 0.0005 or, in some instances, not lower than 0.0007.

Since the double-bond isomerization of isomerization of isoprenol to prenol is an equilibrium reaction, a complete conversion of substances is not achieved in a single pass. Instead, a portion of isoprenol always remains, which unreacted isoprenol is suitably separated from the desired prenol. The unreacted isoprenol may be recycled to the isomerization reaction, or may be directed to other isoprenol-consuming reactions.

Generally, the reactant stream will comprise or consist of a fresh isoprenol stream. The term “fresh isoprenol stream” refers to a stream of isoprenol directly obtained from the purification unit following the isoprenol synthesis, i.e., from a purification unit wherein a crude isoprenol stream from the reaction of isobutene and formaldehyde is purified. The reactant stream may further comprise recycled, unreacted isoprenol, and/or isoprenol from other sources.

Preferably, the reactant stream comprises or consists of a fresh isoprenol stream. Also preferably, the reactant stream comprises or consists of a mixture of unreacted isoprenol stream and a fresh isoprenol stream.

In yet another embodiment, the reactant stream consists of a mixture of the unreacted isoprenol stream, and isoprenol from other sources. Other sources of isoprenol are processes other than the reaction of isobutene and formaldehyde, in which isoprenol is obtained as a by-product or target product, or isoprenol from commercial sources.

The presence of aldehydes, especially formaldehyde and/or prenal in the reactant stream reduces both catalyst activity and selectivity and causes increase in pressure drop and reactor clogging. Besides aldehydes, especially formaldehyde and/or prenal, other impurities which may be present in the reactant stream can cause a decrease in catalyst activity and selectivity. Preferably, the equipment or operations used for maintaining in the reactant stream a certain concentration of aldehydes, preferably formaldehyde and/or prenal, or a certain weight ratio of aldehydes, preferably formaldehyde or prenal to isoprenol is also effective to remove a major portion of these impurities. In preferred embodiments, the concentration in the reactant stream of at least one of the following impurities is kept below the limit indicated, in particular of all of the following impurities:

| | Concentration limit (wt.-%), based |
|---|---|
| Impurity | on the total weight of the stream |
| Isoamylalcohol | 0.05 |
| Prenol | 0.20 |

Compliance with these limits is particularly important when the reactant stream accommodates isoprenol streams from other sources.

Reducing the concentration of aldehydes, preferably formaldehyde and/or prenal in the reactant stream will inherently reduce the weight ratio of aldehydes, preferably formaldehyde and/or prenal to isoprenol in the reactant stream. Therefore, the following applies for reducing the concentration of aldehydes, preferably formaldehyde and/or prenal in the reactant stream as well as reducing the weight ratio of aldehydes, preferably formaldehyde and/or prenal to isoprenol in the reactant stream.

The presence of formaldehyde in the reactant stream is due to two main sources. Formaldehyde may be contained in the isoprenol stream sent to the reactor, that is as an impurity originating from the isoprenol manufacture step. In industrial practice, isoprenol is synthesized from isobutene and formaldehyde. All the formaldehyde that cannot be separated in the purification step following the isoprenol synthesis ends up in the reactant stream.

In addition, formaldehyde is also generated in situ. Part of the isoprenol splits back to isobutene and formaldehyde.

Since most continuous industrial processes operate at single-pass conversion levels of 50 to 60% and with recycling of the unconverted isoprenol, formaldehyde may be present in the recycling stream of unconverted isoprenol, if no steps to purify the stream containing unreacted isoprenol are taken. The recycle stream of unconverted isoprenol has now been found to typically constitute the biggest source of formaldehyde contamination in the reactant stream. The process is generally carried out at partial conversions, for example at conversions of 30 to 70%, preferably 50 to 60%. An unreacted isoprenol stream is separated from the product stream. The unreacted isoprenol stream is recycled, that is, combined with a fresh feed stream comprising isoprenol (a crude isoprenol stream) to provide the reactant stream. The unreacted isoprenol stream comprises isoprenol as a main constituent, but may also comprise prenal, isoprenal, isoamylalcohol, isovaleraldehyde, isovaleric acid, prenol, formaldehyde. It can also contain traces of other C₃ and C₂ aldehydes and acids.

Prenal may be contained in the isoprenol stream sent to the reactor, that is as an impurity originating from the isoprenol manufacture step. The isoprenol stream may further contain traces of ammonia, and/or C₅-oxygenates other than prenal besides formaldehyde and/or prenal. All the prenal and/or other impurities that cannot be separated in the purification step following the isoprenol synthesis ends up in the reactant stream.

Since the double bond isomerization of isoprenol is an equilibrium reaction, conversion is necessarily incomplete. For economic operation of the process, the unconverted isoprenol has to be removed and recycled. Recycling of isoprenol may therefore inadvertently (re) introduce formaldehyde into the isomerization step if no steps to purify the stream containing unreacted isoprenol are taken.

Reducing the concentration of aldehydes, preferably formaldehyde and/or prenal in the reactant stream or reducing the weight ratio of aldehydes, preferably formaldehyde and/or prenal to isoprenol in the reactant stream can be accomplished in several different ways.

In a preferred embodiment, the process includes separating an unreacted isoprenol stream from the prenol containing product stream, optionally removing at least some aldehydes, preferably some formaldehyde and/or prenal from the unreacted isoprenol stream, followed by combining the unreacted isoprenol stream with a fresh isoprenol stream to form the reactant stream.

In another preferred embodiment, the process includes separating an unreacted isoprenol stream from the prenol containing product stream, combining the unreacted isoprenol stream with a crude isoprenol stream containing isoprenol, water and aldehydes, and removing aldehydes, preferably water and aldehydes from the combined stream to form the reactant stream

As mentioned above, the crude isoprenol stream is generally the product stream of an isoprenol production process from which unreacted isobutylene has been removed. This means that formaldehyde removal is accomplished in the purification unit following the isoprenol synthesis. A preferred method of recovering formaldehyde from a crude isoprenol stream to which an unreacted isoprenol stream is admixed, is described in more detail below.

Aldehydes, preferably formaldehyde and/or prenal may be removed from isoprenol streams by a conventional separating method such as distillation, selective adsorption and/or selective reaction.

Removal of aldehydes, preferably formaldehyde and/or prenal by distillation can involve the use of a single distillation column or a train of distillation columns. The towers and columns used may be conventional distillation columns. Suitable types of distillation columns include packed columns, such as columns with random packing or structured packing, plate columns (i.e., tray columns), and mixed columns comprising both packings and trays.

Suitable plate columns may comprise internals over which the liquid phase flows. Suitable internals include sieve trays, bubble cap trays, valve trays, tunnel trays and Thormann® trays, in particular bubble cap trays, valve trays tunnel trays and Thormann® trays. Random packed columns may be filled with a variety of shaped bodies. Heat and mass transfer are improved by enlarging the surface area by means of shaped bodies, which usually have a size in the range of 25 to 80 mm. Suitable shaped bodies include Raschig rings (hollow cylinders), Lessing rings, Pall rings, Hiflow rings and Intalox saddles. The packing materials may be provided in the column in a regular or irregular manner (as bulk material, i.e. loosely filled). Suitable materials include glass, ceramics, metal and plastics.

Structured packings are an advancement of regular packings and have a regularly shaped structure. This allows for the reduction of gas flow pressure loss. Suitable types of structured packings include fabric and metal sheet packings.

Removal of aldehydes, preferably formaldehyde and/or prenal by selective adsorption involves contacting the stream with an adsorbent that exhibits selectivity for low molecular weight aldehydes, especially formaldehyde and/or prenal. Useful adsorbent materials should deliver high selectivity and high adsorption capacity. An additional and critically important requirement is that the adsorbent material should not catalyze or participate in chemical reactions that might lower the recovery of the (iso) prenal and/or render the adsorbent inactive. Adsorbents include ion exchange resins, mesoporous solids, activated carbons, and zeolites.

Removal of aldehydes, preferably formaldehyde and/or prenal by selective reaction involves exposing the stream to reaction conditions under which aldehydes, preferably formaldehyde and/or prenal are (is) selectively reacted to products that are less prone to catalyst deactivation and clogging or to products that can be separated from the stream more easily than aldehydes, preferably formaldehyde and/or prenal.

Preferably, removal of aldehydes, preferably formaldehyde and/or prenal from a stream comprising isoprenol is conducted by distillation, selective adsorption and/or selective reaction, in particular by purification process involving the pressure-swing distillation.

The above described applies for the reducing the concentration of aldehydes other than formaldehyde or prenal and/or of other impurities in the reactant stream as well as reducing the weight ratio of aldehydes other than formaldehyde or prenal to isoprenol.

In case of formaldehyde, difficulties arise from the fact that monomeric formaldehyde (as well as polymeric formaldehyde) forms both hydrates with water and hemiformals with alcohols such as isoprenol, which is the reactant of the isoprenol isomerization and may still remain in the product stream as unreacted reactant. The hydrates and hemiformals of varying formaldehyde polymerization degree have intermingling boiling points. The stability of and the equilibrium between hydrates and hemiformals is temperature-dependent. Formals formed in an upper region of a distillation tower may decompose in the hotter bottom of the tower, which adds additional complexity to the separation task.

In an embodiment, the unreacted isoprenol stream is combined with a crude isoprenol stream containing isoprenol, water and aldehydes, preferably formaldehyde and/or prenal; and removing aldehydes, such as formaldehyde and/or prenal, preferably water and aldehydes, in particular water and formaldehyde and/or prenal, from the combined stream comprises
(i) directing the combined stream to a first low-boiler separation tower operated at a pressure of 1.5 bara or lower, to obtain a first bottoms stream containing isoprenol and aldehydes, preferably prenal and/or formaldehyde, and a first distillate stream containing water and low-boilers;
(ii) directing the first bottoms stream to a second low-boiler separation tower operated at a pressure of 2 bara or higher, to obtain a second distillate stream containing aqueous aldehydes, preferably prenal and/or formaldehyde, and a second bottoms stream containing isoprenol; and
(iii) directing the second bottoms stream to a finishing tower to obtain a bottoms stream containing high-boilers, and the reactant stream as a distillate stream.

In order to permit a first distillation at a temperature below the isoprenol-aldehyde dissociation temperature of the respective aldehyde(s) present, for example for formaldehyde the isoprenol-formaldehyde dissociation temperature and a second distillation at a temperature above the isoprenol-aldehyde dissociation temperature, like the isoprenol-formaldehyde dissociation temperature, the invention envisages two low-boiler separation towers operated at different pressures. Hence, at the relatively low pressure prevailing in the first low-boiler separation tower, a first distillate containing water and low-boilers essentially free of aldehydes, preferably formaldehyde and/or prenal is obtained. At the relatively high pressure prevailing in the second low-boiler separation tower, a virtually all aldehydes, preferably all formaldehyde and/or prenal is separated from the isoprenol. The process of the invention thus allows for obtaining isoprenol essentially free of aldehydes, preferably formaldehyde and/or prenal.

The term “essentially free of aldehydes, preferably formaldehyde and/or prenal” is understood to indicate the absence of significant amounts of aldehydes, preferably formaldehyde and/or prenal in the obtained isoprenol. Thus, the obtained isoprenol preferably comprises less than 0.2 wt.-%, in particular less than 0.15 wt.-%, or less than 0.1 wt.-%, based on the total weight of the obtained isoprenol, of aldehydes, preferably formaldehyde and/or prenal.

Preferably, the crude isoprenol stream is a liquid stream. The liquid stream can be a single-phase liquid stream or a two-phase liquid stream.

The crude isoprenol is directed to a first low-boiler separation tower operated at a pressure of 1.5 bara or lower. Any higher pressure of the crude isoprenol stream is preferably released before the same is directed to the first low-boiler separation tower. The crude isoprenol stream is preferably fed to the first low-boiler separation tower as a side stream, defining a rectifying section above the location of the feed and a stripping section below the location of the feed.

In the first low-boiler separation tower, a first bottoms stream containing isoprenol and aldehydes, preferably formaldehyde and/or prenal, and a first distillate stream containing water and low-boilers are obtained. The term “low-boilers” is understood to refer to organic compounds (other than aldehydes, especially formaldehyde and/or prenal) having a boiling point lower than that of isoprenol, hence a boiling point of lower than about 130° C., at atmospheric pressure. The most common low-boilers are methanol and/or isoprenyl formate formed as by-products during the process.

In a preferred embodiment, the first low-boiler separation tower is operated at a pressure of 1.2 bara or lower, preferably 0.5 bara or lower. The bottoms temperature of the first low-boiler separation tower is preferably in the range of 80 to 135° C., more preferably 90 to 115° C., most preferably 95 to 105° C. The temperature at the top of the first low-boiler separation tower is preferably in the range of 45 to 105° C., more preferably 55 to 80° C.

In a particularly preferred embodiment, the first low-boiler separation tower is operated at a pressure in the range of 0.2 to 0.5 bara, a bottoms temperature in the range of 90 to 115° C. and a temperature at the top in the range of 55 to 80° C.

The first low-boiler separation tower preferably has from 15 to 65 theoretical plates, more preferably from 25 to 40 theoretical plates. In particular, the stripping section of the first low-boiler separation tower preferably has 10 to 25 theoretical plates. The rectifying section of the first low-boiler separation tower preferably has 5 to 40 theoretical plates.

The first bottoms stream preferably comprises 75 to 95 wt.-% of isoprenol, more preferably 80 to 90 wt.-%, based on the total weight of the first bottom stream.

The first distillate is typically withdrawn at the top of the first low-boiler separation tower in gaseous form and condensed to obtain a liquid two-phase stream. The two-phase stream is preferably allowed to phase-separate in a separating vessel to obtain an aqueous phase and an organic phase. The aqueous phase is preferably passed to a wastewater stripping column described below. The organic phase is preferably partially returned to the top of the first low-boiler separation tower as a reflux stream. Another part of the organic phase is preferably discarded from the process to avoid the accumulation of water-insoluble low-boilers in the first low-boiler separation tower.

In a preferred embodiment, at least part of the first distillate stream is directed to a wastewater stripping column to separate low-boilers and entrained isoprenol from water. Preferably, the part of the first distillate stream directed to the wastewater stripping column is an aqueous phase obtained by condensation and phase separation of the first distillate stream, as discussed above.

In the wastewater stripping column, low-boilers are obtained as the low-boiler distillate stream, and wastewater is obtained as a bottoms stream. Both the low-boiler distillate stream and the wastewater bottoms stream are removed from the process, and each stream may be directed to further processing.

Moreover, isoprenol is preferably obtained as a side stream in the wastewater stripping column. The isoprenol side stream is typically a two-phase stream and preferably comprises 15 to 40 wt.-% of isoprenol, more preferably 25 to 35 wt.-%, based on the total weight of the isoprenol side stream. The isoprenol side stream is preferably recycled to the first low-boiler separation tower.

The low-boiler distillate stream preferably comprises 75 to 95 wt.-% of low-boilers, more preferably 80 to 85 wt.-%, based on the total weight of the low-boiler distillate stream. The wastewater bottoms stream preferably comprises less than 1.2 wt.-% of organic matter, more preferably less than 0.6 wt.-%, based on the total weight of the wastewater bottoms stream. The wastewater bottoms stream typically comprises aldehydes, preferably formaldehyde and/or prenal in a concentration of 0.05 to 1.5 wt.-% of aldehydes, preferably formaldehyde and/or prenal, such as 0.3 to 0.9 wt.-%, based on the total weight of the wastewater bottoms stream.

The wastewater stripping column is preferably operated at a pressure of 1.5 bara or lower, preferably 1.1 bara or lower. The bottoms temperature of the wastewater stripping column is preferably in the range of 95 to 110° C., more preferably 97 to 103° C. The temperature at the top of the wastewater stripping column is preferably in the range of 65 to 100° C., more preferably 75 to 85° C.

In a particularly preferred embodiment, the wastewater stripping column is operated at a pressure in the range of 0.95 to 1.1 bara, a bottoms temperature in the range of 97 to 103° C. and a temperature at the top in the range of 75 to 85° C.

The wastewater stripping column preferably has from 6 to 30 theoretical plates, more preferably from 10 to 20 theoretical plates.

The first bottoms stream obtained in the first low-boiler separation tower is directed to a second low-boiler separation tower operated at a pressure of 2 bara or higher. The first bottoms stream is preferably fed to the second low-boiler separation tower as a side stream, defining a rectifying section above the location of the feed and a stripping section below the location of the feed.

In the second low-boiler separation tower, a second distillate stream containing or consisting essentially of aqueous aldehydes, preferably formaldehyde and/or prenal, and a second bottoms stream containing isoprenol are obtained. The second bottom stream further comprises high-boilers. The term “high-boilers” is understood to refer to organic compounds having a boiling point higher than that of isoprenol, i.e. higher than about 130° C., at atmospheric pressure.

In a preferred embodiment, the second low-boiler separation tower is operated at a pressure of 2.5 bara or higher, preferably 2.8 bara or higher, most preferably 2.9 bara or higher. The bottoms temperature of the second low-boiler separation tower is preferably in the range of 160 to 200° C., more preferably 170 to 185° C., most preferably 175 to 180° C. The temperature at the top of the second low-boiler separation tower is preferably in the range of 115 to 160° C., more preferably 125 to 145° C.

In a particularly preferred embodiment, the second low-boiler separation tower is operated at a pressure in the range of 2.9 to 3.5 bara, a bottoms temperature in the range of 175 to 180° C. and a temperature at the top in the range of 130 to 140° C.

The second low-boiler separation tower preferably has from 20 to 60, more preferably from 35 to 60 theoretical plates. In particular, the stripping section of the first low-boiler separation tower preferably has 25 to 45 theoretical plates. The rectifying section of the first low-boiler separation tower preferably has 7 to 20 theoretical plates.

At the top of the second low-boiler separation tower, an offgas is typically obtained. The offgas primarily comprises nitrogen and may comprise traces of isoprenol, formic acid, water, aldehydes, preferably formaldehyde, prenal and/or decomposition gases.

The second bottoms stream preferably comprises 82 to 96 wt.-% of isoprenol, more preferably 87 to 91 wt.-%. The relatively high pressure of the second low-boiler separation tower allows for a high degree of separation of aldehydes, preferably formaldehyde and/or prenal, and isoprenol. Thus, the second bottoms stream preferably comprises at most 0.5 wt.-%, more preferably at most 0.1 wt.-%, even more preferably at most 0.008 wt % of aldehydes, preferably formaldehyde and/or prenal, based on the total weight of the second bottoms stream.

The second distillate stream is an aqueous stream, which preferably comprises 25 to 60 wt.-%, more preferably 40 to 50 wt.-%, in particular 45 to 50 wt.-%, based on the total weight of the second distillate stream, of aldehydes, preferably formaldehyde and/or prenal. The second distillate stream preferably comprises at most 15 wt.-% of isoprenol, more preferably at most 5 wt.-%, based on the total weight of the second distillate stream, of isoprenol.

Owing to the broad condensation curve of the vapor emerging at the top of the second low-boiler separation tower, it is advantageous to use a condenser with liquid recycling. The direct condensation in a quench with liquid circulation is particularly advantageous. Hence, in a preferred embodiment of the process, a quench section is provided downstream, in vapor flow direction, of the rectifying section of the second low-boiler separation tower. The term “vapor flow direction” relates to the direction of the flow of gaseous components in the separation tower, i.e. upwards, towards the top of the tower. The quench section is preferably provided within the second low-boiler separation tower above the rectifying section.

The direct condensation in a quench also mitigates fouling caused by various condensation and polymerization mechanisms of aldehydes, for example formaldehyde that may occur at spots of high local aldehyde concentrations, like local formaldehyde concentrations. To avoid the risk of fouling in the second low-boiler separation tower and downstream processes, in particular in the offgas of the second low-boiler separation tower, the concentration of aldehydes, preferably formaldehyde and/or prenal in the second distillate is preferably no higher than 60 wt.-%, more preferably no higher than 55 wt.-% and in particular no higher than 50 wt.-%, based on the total weight of the second distillate stream.

At the lower end of the quench section, an aqueous liquid is collected. When the quench section is provided within the second low-boiler separation tower, the aqueous liquid may be collected, e.g., at a collecting tray above the rectifying section and beneath of the quench section.

The aqueous liquid is partially circulated into the quench section through a circulation line and partially withdrawn as the second distillate. Suitably, the part of the aqueous liquid circulated into the quench section is circulated into the top of the quench section. Circulation of the aqueous liquid is typically achieved by use of a pump.

The circulation of a part of the aqueous liquid into the quench section allows for cooling of vapors rising through the quench section, and absorption of aldehydes, preferably formaldehyde and/or prenal from the vapors into the aqueous liquid. Thus, aldehydes, preferably formaldehyde and/or prenal is quenched from the vapors rising through the quench section.

Further, the aqueous liquid is partially returned to the rectifying section of the second low-boiler separation tower as a reflux stream. This may be accomplished by a reflux line, or aqueous liquid may be partially returned to the rectifying section as overflow from a collecting tray beneath the quench section.

The mass flow ratio of the reflux stream to the second distillate is preferably in the range of 2:1 to 10:1, more preferably in the range of 3:1 to 7:1. In a preferred embodiment, the aqueous liquid is cooled before being circulated into the quench section. Preferably, the part of the aqueous liquid withdrawn as the second distillate is a partial stream of the cooled aqueous liquid.

The temperature of the aqueous liquid collected at the lower end of the quench section is preferably in the range of 80 to 140° C., more preferably 125 to 135° C. The temperature of the cooled aqueous liquid circulated into the quench section is preferably 10 to 80° C. below the temperature of the aqueous liquid collected at the lower end of the quench section. This allows for an energetically favorable process.

The hot aqueous liquid withdrawn at the lower end of the quench section lends itself to heat-integration. In a suitable embodiment, it is heat-exchanged with the stream of crude isoprenol flowing into the first low-boiler separation tower before being circulated into the quench section.

In one embodiment, a scrubbing section is provided downstream, in vapor flow direction, of the quench section and water is introduced at the top of the scrubbing section. Preferably, the scrubbing section is provided within the second low-boiler separation tower above the quench section. The scrubbing section allows for maintaining the aldehydes, preferably formaldehyde and/or prenal concentration in the second distillate below the critical concentrations described above and thus to avoid depositions for example paraformaldehyde deposition in, e.g., offgas lines.

The mass flow ratio of the water introduced at the top of the scrubbing section to the first bottoms stream obtained in the first low-boiler separation tower is typically in the range of 0.01:1 to 0.06:1 more preferably in the range of 0.015:1 to 0.03:1.

The second bottoms stream is directed to a finishing tower, in which pure isoprenol is obtained as a distillate stream. High-boilers are withdrawn via a bottoms stream. As the second bottoms stream comprises essentially no aldehydes, preferably no formaldehyde and/or prenal, the separation task of the finishing tower is significantly less complex than in cases where formaldehyde separation is less efficient in the low-boiler separation section.

The term “essentially no aldehydes, preferably no formaldehyde and/or prenal” is understood to indicate the absence of significant amounts of aldehydes, preferably formaldehyde and/or prenal in the obtained isoprenol. Thus, the obtained isoprenol preferably comprises less than 0.05 wt.-%, preferably less than 0.01 wt.-%, based on the total weight of the second bottoms stream, of aldehydes, preferably formaldehyde and/or prenal.

The pure isoprenol distillate stream preferably at least 97.0 wt.-% of isoprenol, more preferably 98.0 wt.-%, such as 98.1 to 99.5 wt.-%, based on the total weight of the pure isoprenol distillate stream. The high-boiler bottoms stream preferably comprises 90 to 99.9 wt.-% of high-boilers, more preferably 99 to 99.8 wt.-%, based on the total weight of the high-boiler bottoms stream. Preferably, the high-boiler bottoms stream comprises less than 0.2 wt.-% of aldehydes, preferably formaldehyde and/or prenal, such as less than 0.05 wt.-%, based on the total weight of the high-boiler bottoms stream, of aldehydes, preferably formaldehyde, and/or prenal.

In a preferred embodiment, the finishing tower is operated at a pressure of 0.5 bara or lower, preferably 0.25 bara or lower. The bottoms temperature of the first low-boiler separation tower is preferably in the range of 130 to 190° C., more preferably 150 to 170° C. The temperature at the top of the finishing tower is preferably in the range of 60 to 90° C., more preferably 65 to 85° C.

In a particularly preferred embodiment, the finishing tower is operated at a pressure in the range of 0.05 to 0.2 bara, a bottoms temperature in the range of 150 to 170° C. and a temperature at the top in the range of 65 to 85° C.

The finishing tower preferably has from 6 to 40 theoretical plates, more preferably from 10 to 20 theoretical plates.

Unreacted isoprenol from the isomerization of isoprenol to prenol may be used, i.e. directed as feed to an oxidative dehydrogenation step of isoprenol to obtain a stream comprising prenal and/or isoprenal, as described in the following.

### Third Aspect—Providing Prenal and/or Isoprenal

In addition, the invention relates to the preparation of prenal and/or isoprenal, comprising
a) providing isoprenol as described above via steps a-i) and a-ii) according to the first aspect, and b) optionally, isomerizing isoprenol obtained in step a) to obtain prenol as described above by bringing a reactant stream comprising isoprenol into contact with at least one heterogeneous isomerization catalyst, preferably in the presence of hydrogen, according to the second aspect, and providing prenal by at least one of c-i) and c-ii):
c-i) subjecting isoprenol obtained in step a) as described above to oxidative dehydrogenation so as to obtain prenal and/or isoprenal by bringing a reactant stream comprising isoprenol into contact with at least one heterogeneous oxidative dehydrogenation catalyst, in the presence of molecular oxygen, and optionally isomerizing at least part of the isoprenal to prenal; and
c-ii) oxidizing prenol obtained in step b) as described above so as to obtain prenal by bringing a reactant stream comprising prenol into contact with at least one oxidant and at least one oxidation catalyst, preferably in the presence of a liquid phase.

The isoprenol obtained as described above is converted to prenal, involving isomerization and an oxidative dehydrogenation in any order. Thus, it is possible to first isomerize isoprenol to prenol, and subsequently oxidize prenol to prenal; or, to first oxidatively dehydrogenate isoprenol to isoprenal, and optionally isomerize at least part of the isoprenal to prenal.

### Third Aspect—Oxidizing Prenol to Prenal

The prenol obtained as described above may be oxidized so as to obtain prenal by bringing a reactant stream comprising prenol into contact with at least one oxidant and at least one oxidation catalyst, preferably in the presence of a liquid phase.

Suitable oxidants include hydrogen peroxide and oxygen, in particular oxygen.

The oxidation is preferably carried out in the presence of a liquid phase and with oxygen as the oxidant. The liquid phase preferably comprises at least 25 wt.-% of water, more preferably at least 50 wt.-% of water or at least 70 wt.-% of water, based on the total weight of the liquid phase, determined at a temperature of 20° C. and a pressure of 1 bar. It has been found that these conditions allow for a simple and efficient process for preparing prenal from prenol.

The oxidation is typically carried out in the presence of at least one oxidation catalyst selected from the group consisting of platinum, palladium and gold. Preferably, the at least one oxidation catalyst comprises platinum. In a preferred embodiment, the at least one oxidation catalyst is a supported catalyst.

The oxidation is suitably carried out at a temperature of 20 to 100° C., preferably, 25 to 80° C., in particular 30 to 70° C., in particular 35 to 50° C. In another embodiment the oxidation is carried out at a temperature of 20 to 70° C. The oxidation is suitably carried out under a partial pressure of oxygen between 0.2 and 8 bar.

Further details of the oxidation reaction may be found in WO2023/222895 A1, which herewith is incorporated by reference in its entirety.

### Oxidative Deyhdrogenation of Isoprenol

Oxidative dehydrogenation of isoprenol typically comprises bringing a reactant stream, in particular a gaseous reactant stream, comprising isoprenol into contact with at least one heterogeneous oxidative dehydrogenation catalyst, in particular at least one silver-containing heterogeneous oxidative dehydrogenation catalyst, in the presence of molecular oxygen. The at least one heterogeneous catalyst may consist of an inert support having a smooth surface having an active layer of silver. Alternatively, massive (full-metal) silver bodies may be used.

In an embodiment, the non-reacted isoprenol from the isomerization of isoprenol to prenol, which corresponds to the step b), is used as feed to the dehydrogenation step.

Hence, in an embodiment, the process includes separating an unreacted isoprenol stream from a prenol containing product stream obtained in step b) and directing the unreacted isoprenol stream at least partially to step c-i).

In an embodiment, oxidative dehydrogenation is carried out by passing the isoprenol through a plurality of reaction tubes of a shell-and-tube heat exchange reactor comprising
a shell-side heat exchange passage for circulating a heat transfer medium and a reaction passage comprising the plurality of reaction tubes;
an inlet for introducing the reactant stream to the reaction passage; and
an outlet from the reaction passage for recovering an effluent stream from the reaction tubes;
   wherein the reaction tubes comprise
   a reactant pre-heating zone adjacent to the inlet, and
   a reaction zone downstream of the reactant pre-heating zone, the reaction zone having a catalytically active wire matrix insert having silver at least on a part of its surface.

The term “reactant pre-heating zone” denotes a section of the reaction tube, i.e. a section inside the reaction tube, where essentially no catalytic oxidative dehydrogenation reaction occurs and where the gaseous stream through the reaction tubes is heat-exchanged via the tube wall with the circulating heat transfer medium. The pre-heating zone upstream of the reaction zone involves net heat flow into the reaction tube and ensures that the reactant stream is sufficiently heated up to a temperature close to or at the reaction temperature when it reaches the reaction zone.

Upon contact with the catalytic surface, the oxidative dehydrogenation reaction immediately starts. Otherwise, in the event when a “cold” reactant stream reaches the catalytic surface such that the reaction onset temperature of the reaction is not reached, coke formation may occur. Less coke formation advantageously leads to a prolonged reactor operation without the necessity of burning off the coke from the catalytic surface.

Preferably, the reactant pre-heating zone is adapted to allow for laminar flow of the reactant inside the reactant pre-heating zone. This means, the reactant pre-heating zone is devoid of any obstacles to the reactant flow that triggers a laminar-to-turbulent flow transition. Hence, the reactant pre-heating zone preferably has an essentially free cross section, i.e. the pre-heating zone is empty.

In the case of an “essentially free cross section”, the reactant pre-heating zone may be empty. Alternatively, the reactant pre-heating zone may accommodate fixtures made of a material having zero or limited catalytic activity, which fixtures have a negligible cross-section in a plane perpendicular to the longitudinal axis of the reaction tube. Said fixtures may be attached to the catalytically active wire matrix which is present in the reaction zone and allow to easily place said wire-matrix insert into or remove the same from the reaction zone. For example, the negligible mounting may be a stainless steel wire or rod.

This setup allows for heating up only the portion of the entire reactant stream that travels near the hot reaction tube wall. Consequently, the portion of the reactant stream flowing in the center of the reaction tube is not heated to the reaction temperature and blind reactions of the unstable starting materials are thus reduced or even avoided. A “blind reaction” is an unselective oxidative reaction that occurs in the absence of the catalyst. Once the reactant stream reaches the reaction zone, the oxidative dehydrogenation reaction is initiated. Due to the exothermic nature of this reaction, energy is released and the remainder of the reactant stream is rapidly heated to the reaction onset temperature, and the reaction proceeds. This fast heat up of the predominant part of the reaction mixture reduces unwanted side-reactions and thus leads to an increased selectivity.

Alternatively, the reactant pre-heating zone may have a wire matrix insert having zero or limited catalytic activity. The wire matrix insert may reduce or eliminate temperature gradients without creating any obstruction to flow that would promote turbulent flow characteristics. A wire matrix insert is considered as having zero catalytic activity (or in other words, as being “inert”) if it does not catalyze the gas-phase partial oxidation reaction in question to a significant degree, and the chemical composition of a stream passing the wire matrix insert does not change significantly. Similarly, a matrix insert is considered as having limited catalytic activity if its catalytic activity is less than the activity of a reaction zone. In an embodiment, the wire matrix insert having zero or limited catalytic activity is made of an inert material, preferably stainless steel.

Herein, the term “reaction zone” denotes a region of the reaction tube where the catalytic gas-phase partial oxidation reaction occurs. The reaction zone comprises a catalytically active wire matrix insert having at least on a part of its surface a catalytically active precious metal. Due to the more open structure of the wire matrix contained in the reaction zone as compared to a packing of individual elements, a larger proportion of the reaction heat is discharged to the reaction tube wall by radiation and does not have to be dissipated by the reactant stream. Due the unique flow characteristic of the reactant stream through the reaction tube with the wire matrix insert in place, heat transfer via the tube wall is improved. Formation of prominent hotspots can be avoided. This in turn, avoids deposition of organic constituents of the reactant stream on the surface of the active catalyst material with concomitant pressure drop. Overall, less regular maintenance in the form of regeneration and/or replacement of the catalyst is required. The number of annual operating hours can be increased and the existing production capacities can be fully utilized, reducing operation cost and increasing profit.

In contrast to individually present catalyst bodies, the wire matrix inserts can be formed contiguously, or in one piece. Hence, placing the wire matrix inserts in the catalyst containment region of the reaction tubes, and removal therefrom is much facilitated.

The “reaction zone” may be comprised of a single contiguous reaction zone. Alternatively, the reaction zone may comprise an alternating series of regions having catalytically active wire matrix inserts and regions having an essentially free cross section or having wire matrix inserts having zero or limited catalytic activity.

A “wire matrix insert” is understood to be a self-supporting skeletal-like structure made of coiled, bent or crimped metal wire which is adapted to be inserted into a reaction tube of a shell-and-tube reactor. The wire matrix insert has a more voluminous structure than a longitudinal wire.

A fixture such as a stainless steel wire or rod may be attached to the wire matrix insert which allows for easily placing the wire-matrix insert into or removing the same from the reaction zone.

In an embodiment, the catalytically active wire matrix inserts comprise an elongated core having a plurality of wire loops extending from the elongated core, wherein the wire loops are longitudinally arranged and helically shifted, that is, neighboring wire loops have an angular offset. The loops may be formed by helically bending the wire over the length of the wire matrix insert. In view of the ease of manufacture, the elongated core preferably comprises at least two longitudinal core wire members, which are twisted around each other to form core wire windings, and the wire loops are accommodated in the core wire windings.

The wire loops may be formed from one wire, or more than one intertwined wire, preferably 4 intertwined wires.

The wire matrix insert comprised in the reaction zone has silver at least on a part of its surface a catalytically active precious metal. The wire constituting the wire loops may be a massive silver wire, or a wire coated with silver. The core wire may be made of brass alloys, or high-grade steels. The coating layer of silver superimposed on the surface of the core has a thickness of, e.g., 10 μm. In general, however, a massive silver wire has better service life and is preferred. If the wire loops are formed from more than one intertwined wire, at least one of the intertwined wires is made of a massive silver wire, or a wire coated with silver while the other intertwined wires can be made of an inert material.

A silver wire which is of the same composition throughout its cross section and comprises at least 92.5 wt.-% Ag can suitably be used. The silver wire is helically bent to form wire loops, and combined with at least two longitudinal core wire members, which are twisted around each other to form core wire windings, and the wire loops are accommodated in the core wire windings. The longitudinal core wire members can also be silver wire or inert metal wire.

In a preferred embodiment, the catalytically active wire matrix inserts comprise an elongated core having a plurality of wire loops extending from the elongated core, wherein the wire loops are longitudinally arranged and helically shifted, and the wire loops comprise a massive silver wire.

Further details of oxidative dehydrogenation carried out by passing the isoprenol through a plurality of reaction tubes of a shell-and-tube heat exchange reactor as described above may be found in WO2023/241952 A1, which herewith is incorporated by reference in its entirety.

When isoprenol is subjected to oxidative dehydrogenation, it may be favorable to maintain in the reactant stream a weight ratio of aldehydes, preferably prenal and/or formaldehyde to isoprenol of less than 0.04, preferably less than 0.03, in particular less than 0.02, or less than 0.01. In another embodiment, the weight ratio of aldehydes, preferably prenal and/or formaldehyde to isoprenol is maintained at less than 0.002, or less than 0.001 and optionally at least 100 ppm.

The weight ratio of aldehydes, preferably prenal and/or formaldehyde to isoprenol in the reactant stream may be maintained at a certain level or less. Reducing the weight ratio of aldehydes, preferably prenal and/or formaldehyde to isoprenol in the reactant stream beyond a certain point, however, reaches a point of rapidly diminishing return. Aldehydes, preferably prenal and/or formaldehyde removal involves additional equipment and operating costs. An economic balance must be taken between the improvement due to reducing the ratio and the cost of maintaining such a ratio. Hence, the weight ratio of aldehydes, preferably prenal and/or formaldehyde to isoprenol is preferably not lower than 0.0005. In an alternative embodiment the weight ratio is not lower than 0.005.

Reactor clogging and pressure drop increase are significantly affected by the presence of aldehydes, preferably prenal and/or formaldehyde in the reactant stream. Catalyst-fouling reactions of condensation and polymerization are believed to be the principal reactions involved in carbon or coke formation on the catalyst. It is thought that this carbon formation involves thermal condensation of aldehydes, preferably prenal and/or formaldehyde or of aldehydes, preferably prenal and/or formaldehyde with the olefinic hydrocarbons isoprenol and (iso) prenal. In the presence of the catalyst, the primary condensation products tend to undergo dehydrogenation and polymerization type reactions and to settle on the catalyst and undergo further dehydrogenation and decomposition until carbonaceous deposits are formed.

The process of the invention according to the third aspect may satisfy the following condition 1), and preferably the following condition 2), or the process meets at least one of the following conditions 1) and 2):
1) Step c-i) is characterized by maintaining in the reactant stream a weight ratio of aldehydes to isoprenol of less than 0.04.
2) Step b) is characterized by maintaining in the reactant stream a concentration of aldehydes of less than 0.5% by weight, preferably less than 0.4% by weight, in particular less than 0.3% by weight, or less than 0.25% by weight, based on the total weight of the reactant stream, and, optionally, the concentration of aldehydes in the reactant stream is not lower than 10 ppm, preferably not lower than 25 ppm, in particular not lower than 50 ppm, or not lower than 100 ppm, based on the total weight of the reactant stream.

Reducing the weight ratio of aldehydes, preferably prenal and/or formaldehyde to isoprenol in the reactant stream can be accomplished in several different ways. In an embodiment, aldehydes, preferably prenal and/or formaldehyde, are removed from the unreacted isoprenol stream prior to combining the unreacted isoprenol stream with the crude isoprenol stream.

In an embodiment, the unreacted isoprenol stream is combined with the crude isoprenol stream and aldehydes, preferably prenal and/or formaldehyde is removed from the combined stream.

Aldehydes, preferably prenal and/or formaldehyde, may be removed from isoprenol streams by a conventional separating method such as distillation, selective adsorption and or selective reaction, in particular by the purification process involving the pressure-swing distillation as described above.

### Pre-Treating (Iso) Prenol by Nitrogen Removal Prior to Oxidative Deyhdrogenation or Oxidation

Prior to contacting with the at least one oxidative dehydrogenation catalyst or with the at least one oxidation catalyst, respectively, the (iso) prenol may advantageously be treated to remove organically bound nitrogen from the (iso) prenol by contacting the (iso) prenol with a weakly acidic solid adsorbent. In other words, the (iso) prenol may be depleted of organically bound nitrogen by this process.

The term “organically bound nitrogen” is intended to denote any compound containing at least one nitrogen atom directly bound to one or more carbon atoms. For example, such compounds containing at least one nitrogen atom may be selected from amines, such as ethylamine, trimethylamine, aniline, pyridine or piperidine. An amine particularly significant in practice is hexamethylenetetramine (urotropin). (Iso) prenol may comprise about 5 to 30 ppm of organically bound nitrogen.

The weakly acidic solid adsorbents have been found to be capable of adsorbing organically bound nitrogen in the presence of abundant (iso) prenol while not interfering with the reactive carbon-carbon double bond.

The weakly acidic adsorbent may include an adsorbent material having sufficient acidity to adsorb the organically bound nitrogen from the (iso) prenol. In an embodiment, the solid adsorbent is a crosslinked resin having phosphonic functional groups. Preferably, the resin polymer is a vinyl aromatic copolymer, preferably crosslinked polystyrene and more preferably a polystyrene divinylbenzene copolymer. Other polymers having a phosphonic functional group may also be used. Preferably, the crosslinked resin having phosphonic functional groups is of the macroporous type. A preferred solid adsorbent is Purolite S956.

The resin is typically used in bead form and loaded into a column. The (iso) prenol is passed through the column, contacting the resin beads. During contact, the organically bound nitrogen in the (iso) prenol reacts with the functional group and an exchange occurs where a proton is transferred to the nitrogen and an ionic bond is formed to the anionic site of the resin. Contact is maintained until a threshold level is reached i.e. the breakthrough concentration. At this breakthrough point, the process reaches an equilibrium where additional organically bound nitrogen cannot be removed effectively. The flow is halted and the column is backwashed with water, preferably deionized or softened water. By flowing in reverse, the resin is fluidized and solids captured by the beads are loosened and removed.

In another embodiment, the solid adsorbent is a silica-alumina hydrate. Numerous silica-alumina catalyst compositions and processes for their preparation are described in the patent literature, see, e.g., U.S. Pat. No. 4,499,197.

Preferably, the alumina content of the silica-alumina hydrate is from about 10 to about 90 wt.-% of Al₂O₃. The preferred range of alumina content is from about 30 to about 70 wt.-% of Al₂O₃.

The introduction of silicon dioxide into aluminum oxide leads to the introduction of acidic centers. The number of acidic centers can be controlled by the amount of introduced silicon dioxide. The number of acidic centers increases with the amount of introduced silicon dioxide up to a maximum number of acidic centers, and decreases again with a further increasing amount of silicon dioxide after having reached the maximum number of acidic centers.

Examples of commercially available silica-alumina hydrates are Siral® available from Sasol Germany Gmbh, Hamburg, Germany. Siral® is based on orthorhombic aluminum oxide hydroxide (boehmite; AlOOH) and doped with SiO₂. Various Siral® grades having different ratios of Al₂O₃ to SiO₂ are available: Siral 1 (Al₂O₃/SiO₂=99/1), Siral 5 (Al₂O₃/SiO₂=95/5), Siral 10 (Al₂O₃/SiO₂=90/10), Siral 20 (Al₂O₃/SiO₂=80/20), Siral 28M (Al₂O₃/SiO₂=72/28), Siral 30 (Al₂O₃/SiO₂=70/30), Siral 40 (Al₂O₃/SiO₂=60/40). Siral 40 is especially preferred.

In an embodiment, the (iso) prenol is passed over a bed of the weakly acidic solid adsorbent. Suitably, said step of “passing over a bed” denotes that a layer (“bed”) of the weakly acidic solid adsorbent is provided in a customary reaction vessel known to the skilled person which may preferably be equipped with a stirring device, e.g. in a stirred-tank reactor. The (iso) prenol is then introduced into the reaction vessel and guided through the same in a manner that it gets into contact with the weakly acidic solid adsorbent.

Alternatively, the weakly acidic solid adsorbent may be provided in a reaction tube, e.g. of a tubular reactor and the (iso) prenol then continuously flows through said reaction tube(s) while getting into contact with the weakly acidic solid adsorbent.

In an alternative embodiment, the (iso) prenol comprises, after contacting the alcohol stream with a weakly acidic solid adsorbent, less than 2 ppm of organically bound nitrogen. Herein, “ppm” denotes wt.-ppm of compounds incorporating organically bound nitrogen, relative to the total weight of the (iso) prenol.

For example, the content of organically bound nitrogen in the (iso) prenol may be determined by Kjeldahl analysis. Alternatively, an oxidative combustion method with a chemiluminescence detector according to DIN 51444 may be used.

### Fourth Aspect—Preparation of Citral

In addition, the invention relates to the preparation of 3,7-dimethyl-octa-2,6-dienal (citral), comprising the steps of a) providing isoprenol as described above, b) isomerizing the obtained isoprenol to obtain prenol as described above, and c) providing prenal by at least one of steps c-i) and c-ii) as described above, and further
d) condensing prenol obtained in step b) with prenal obtained in step c) to obtain diprenyl acetal of prenal; and
e) subjecting diprenyl acetal of prenal obtained in step d) to cleaving conditions to obtain citral.

In particular, 3,7-dimethyl-octa-2,6-dienal (citral) can be prepared by a process comprising the steps of:
condensing the prenal with prenol in the presence of at least one catalyst in a reaction column while withdrawing an acetal fraction comprising the diprenyl acetal of prenal from the reaction column;
subjecting the acetal fraction in a cleaving column to cleaving conditions in the presence of at least one catalyst while withdrawing from the cleaving column a cleaving fraction containing at least one of prenyl (3-methyl-butadienyl) ether and 2,4,4-trimethyl-3-formyl-1,5-hexadiene, and optionally containing citral; and
reacting the cleaving fraction in a plug-flow type reactor to obtain citral.

The overall reaction sequence is illustrated by the reaction scheme below.

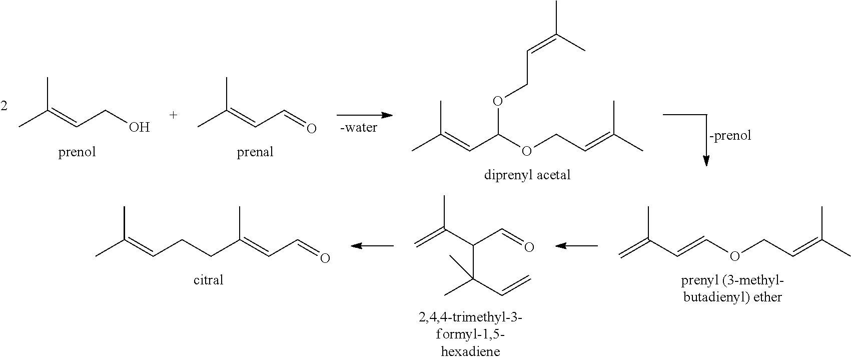

The unsaturated acetal 3-methyl-2-butenal-diprenyl acetal (herein referred to as “diprenyl acetal of prenal” or “diprenyl acetal”) is formed from prenol and prenal using at least one catalyst. For this purpose, prenal may be reacted together with prenol in the presence of catalytic amounts of at least one acid and with separation of the water formed during the reaction in a reaction column.

It has been found that when the conversion rate of diprenyl acetal of prenal is driven to full conversion, the concentration of by-products increases sharply. Accordingly, it is preferred that the conversion rate of diprenyl acetal of prenal is maintained at above 90% and below 100%. Preferably, the conversion rate of diprenyl acetal of prenal in step b) is maintained equal to or below 99.5%, preferably equal to or below 99%, such as equal to or below 98%, or equal to or below 97.5%, or equal to or below 97%. Preferably, the conversion rate of diprenyl acetal of prenal is maintained above 91%, such as above 92%, or above or 93%, or above 94%, or above 95%. In suitable embodiments, the conversion rate of diprenyl acetal of prenal in is above 94% and equal to or below 99%, such as above 95% and equal to or below 98%. Lower conversion rates will render the process economically unprofitable or will otherwise necessitate recovery and recycling of unreacted diprenyl acetal. Complete conversion is however undesirable as it results in a drop of yield of citral building blocks and increasing by-products-formation. The conversion rate is governed by various parameters including cleaving temperature, nature and concentration of the catalyst(s) and residence time in the cleaving column.

The resulting 3-methyl-2-butenal diprenyl acetal (diprenyl acetal) is cleaved in the presence of at least one catalyst in a cleaving column with elimination of 3-methyl-2-buten-1-ol (prenol) to give prenyl (3-methylbutadienyl) ether. Claisen rearrangement of the obtained prenyl (3-methylbutadienyl) ether yields 2,4,4-trimethyl-3-formyl-1,5-hexadiene which subsequently undergoes Cope rearrangement yielding 3,7-dimethyl-2,6-octadienal (citral).

Cleaving is carried out in the presence of at least one catalyst, preferably an acid catalyst. The catalyst can be a single catalytic species or a combination of two or more different catalytic species. Suitable acid catalysts are selected from non-volatile protic acids such as sulfuric acid, p-toluenesulfonic acid and phosphoric acid. In an embodiment, the catalyst comprises phosphoric acid. In a preferred embodiment, the concentration of the phosphoric acid in the bottoms of the cleaving column is maintained above 100 ppm and below 1500 ppm, preferably above 200 ppm and below 1000 ppm. Higher concentrations of (acid) catalyst may result in reduced yields of citral building blocks.

Condensation of prenol with prenal is carried out in the presence of at least one catalyst, preferably an acid. The catalyst can be a single catalytic species or a combination of two or more different catalytic species. In an embodiment, the catalyst in is nitric acid. Preferably, the concentration of the nitric acid is below 500 ppm, more preferably in the range of from 100 to 300 ppm, relative to the total amount of the starting materials prenol and prenal. Lower amounts of (acid) catalyst may result in a low conversion in the reaction column. Higher amounts of (acid) catalyst may disadvantageously result in increased formation of by-products and in decreased selectivities.

Preferably, the acetal fraction is continuously subjected to cleaving conditions in a cleaving column. “Cleaving conditions” denotes reaction conditions selected such that the diprenyl acetal contained in the acetal fraction is cleaved to prenyl (3-methylbutadienyl) ether which may subsequently rearrange to 2,4,4-trimethyl-3-formyl-1,5-hexadiene and citral.

The acetal fraction comprises diprenyl acetal as a main constituent. The acetal fraction does not necessarily need to consist of pure diprenyl acetal, but may also comprise prenol, prenal and citral building blocks.

Cleaving is carried out in the presence of at least one catalyst, preferably at least one acid catalyst. Suitable acid catalysts are selected from non-volatile protic acids such as sulfuric acid, p-toluenesulfonic acid and phosphoric acid.

Suitably, the continuous cleaving in the cleaving column may be carried out in the lower part or the sump of the distillation column acting as cleaving column. Preferably, the acetal fraction and/or the catalyst(s) are introduced into the lower part of the distillation column, into the sump of the distillation column or into the evaporator of the distillation column.

If desired, a high-boiling inert compound can be introduced into the sump of the cleaving column in order to ensure a minimum filling level of the sump and the evaporator. Suitable high-boiling inert compounds are selected from liquid compounds which are inert under the reaction conditions and have a higher boiling point than citral and diprenyl acetal. For example, the high-boiling inert compounds may be selected from hydrocarbons such as tetradecane, pentadecane, hexadecane, octadecane, eicosane; or ethers such as diethylene glycol dibutyl ether; white oils; kerosene oils; or mixtures thereof.

Suitably, the distillation conditions are selected such that the diprenyl acetal is predominantly retained in the lower part or the sump of the distillation column. During the cleaving reaction, a cleaving fraction is continuously withdrawn from the cleaving column, the cleaving fraction containing at least one of prenyl (3-methyl-butadienyl) ether and 2,4,4-trimethyl-3-formyl-1,5-hexadiene, and optionally containing citral. For the ease of reference, prenyl (3-methyl-butadienyl) ether, 2,4,4-trimethyl-3-formyl-1,5-hexadiene and citral are collectively referred to as “citral building blocks”. This is because the former are intermediates on the reaction route to citral and can be converted into citral in the subsequent passage through the plug-flow type reactor.

Additionally, the prenol formed during the cleaving reaction may be continuously removed from the reaction mixture, generally at the top of the cleaving column.

The cleaving fraction together with the formed prenol may be withdrawn at the top of the distillation column.

Alternatively and preferably, it is also possible to withdraw the cleaving fraction in liquid or vaporous form at a side draw of the distillation column.

The cleaving fraction may be reacted in a plug-flow type reactor to obtain citral. To this end, the cleaving fraction is guided through the plug-flow type reactor at a suitable temperature for carrying out the rearrangement reaction(s) yielding citral. By employing a combination of a highly back-mixed cleaving column and a plug-flow reactor, it is possible to increase the selectivity and the yield of the cleaving reaction. All of the catalyst(s) required for the cleaving reaction is/are preferably introduced into the cleaving column and preferably, no catalyst is introduced into the plug-flow reactor.

In an embodiment, prenol eliminated in the cleaving reaction is recycled to the condensation reaction. This allows for improved yields to be achieved in the process of the invention.

In particular, the inventive process may comprise recycling prenol obtained in step e) to step d);
wherein the concentration of 2,4,4-trimethyl-3-formyl-1,5-hexadiene of the prenol recycled from step e) into step d) is controlled such that the concentration of 2,4,4-trimethyl-3-formyl-1,5-hexadiene in step d) is below 1 wt.-%, relative to the total weight of prenol and prenal; and wherein the concentration of citral of the prenol recycled from step e) into step d) is controlled such that the concentration of citral in step d) is below 1 wt.-%, relative to the total weight of prenol and prenal.

### Further Conversion to Menthol or Linalool

The thus obtained citral is a useful intermediate for, e.g., menthol or linalool.

Menthol may be prepared from citral via a process comprising the steps of
catalytic hydrogenation of citral obtained by using the above described processes to obtain citronellal;
cyclization of citronellal to obtain isopulegol in the presence of at least one acidic catalyst; and
catalytic hydrogenation of isopulegol to obtain menthol.

The overall reaction sequence is illustrated by the reaction scheme below.

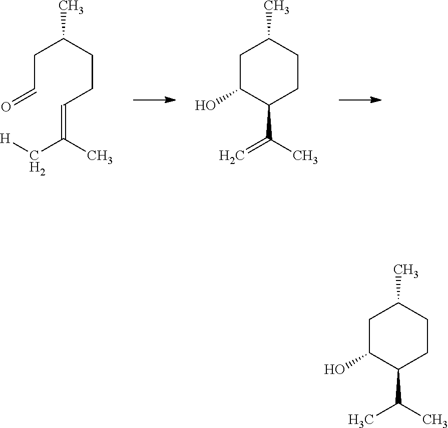

The hydrogenation of citral to obtain citronellal may be achieved by hydrogenation in the presence of a rhodium-phosphine catalyst.

The cyclization of citronellal to isopulegol may be achieved by cyclization in the presence of at least one Lewis-acidic aluminum-containing catalyst, such as a bis(diarylphenoxy) aluminum compound, which may be used in the presence of an auxiliary, such as a carboxylic anhydride. The isopulegol may be recovered from the catalyst-containing reaction product by distillative separation to give an isopulegol-enriched top product and an isopulegol-depleted bottom product. From the bottom product, the at least one catalyst may be regenerated. The isopulegol obtainable in this way by the cyclization of citronellal can be further purified by suitable separating and/or purification methods, in particular by crystallization, and be at least largely freed from undesired impurities or by-products.

The hydrogenation of isopulegol may be achieved by hydrogenation in the presence of at least one heterogeneous nickel-containing catalyst, preferably at least one heterogeneous nickel- and copper-containing catalyst.

Further details regarding the reaction sequence from citral to menthol may be found in US2013/46118A1, which is incorporated by reference herein.

In one aspect, the invention thus relates to an improved process for the preparation of menthol by producing citral using the above processes and then producing menthol from the citral. Menthol may be prepared as described herein or by other methods known in the art.

Linalool may be prepared from citral via a process comprising catalytic hydrogenation of citral to obtain nerol and/or geraniol, and isomerization thereof.

The hydrogenation of citral to obtain nerol and/or geraniol may be achieved by hydrogenation in the presence of at least one supported ruthenium, rhodium, osmium, iridium or platinum catalyst, preferably a ruthenium catalyst supported on carbon black, ruthenium/iron catalyst supported on carbon, comprising 0.1 to 10% by weight of ruthenium and 0.1 to 5% by weight of iron. Reference is made to EP1318128A2 and WO2017/060243.

The isomerization of nerol and/or geraniol to obtain linalool may be achieved by isomerization in the presence of a tungsten catalyst, especially a dioxotungsten (VI) complex, very especially a dioxotungsten (VI) complex of the general formula

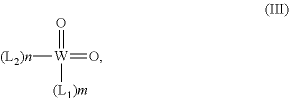

wherein L₁ and L₂ are independently of each other a ligand selected from the group consisting of the aminoalcohols, the aminophenols and mixtures thereof; and m and n are each 1 or 2. Further details regarding the isomerization of geraniol may be found in WO03/048091 and WO03/047749.

In one aspect, the invention thus relates to an improved process for the preparation of linalool by producing citral using the above processes and then producing linalool from the citral. Linalool may be prepared as described herein or by other methods known in the art.

### Further Conversion to Vitamin A

The obtained citral is also a useful intermediate for the synthesis of vitamin A.

Vitamin A acetate may be prepared from citral via the reaction sequence illustrated by the reaction scheme below.

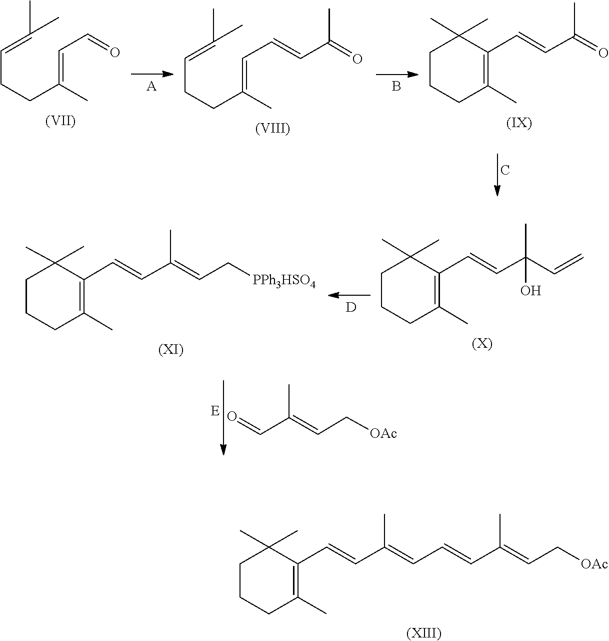

Citral (VII) can be converted into pseudoionone (VIII) in reaction step A. Said pseudoionone can be reacted in synthetic step B to obtain β-ionone (IX), which is further transformed into β-vinylionol of formula (X). Phosphorylation of B-vinylionol of formula (X) can yield the C₁₅-salt of formula (XI), which upon reacting it with the C₅-acetate of formula (XII) can yield vitamin A acetate of formula (XIII).

Accordingly, Vitamin A acetate may be prepared from citral via a process comprising the steps of
converting Citral (VII) obtained by using the above described processes into pseudoionone (VIII),
reacting pseudoionone (VIII) to obtain β-ionone (IX),
transforming β-ionone (IX) into β-vinylionol of formula (X),
phosphorylation of B-vinylionol of formula (X) to yield the C₁₅-salt of formula (XI), and
reacting the C₁₅-salt of formula (XI) with the C₅-acetate of formula (XII) to yield vitamin A acetate of formula (XIII).

Reaction step A can be realized in the presence of a base selected form metal hydroxides, in particular alkali metal hydroxides and earth alkali metal hydroxides. Said base acts as a catalyst and can be added in one or several portions as e.g. disclosed in EP0062291A1 and WO2004/041764A1.

Cyclisation of pseudoionone (VIII) into B-ionone (IX) in step B is realized in the presence of an acid, preferably in the presence of a mineral acid. A method of realizing step B is disclosed in EP0133 668A2 and in U.S. Pat. No. 3,840,601. The vinylionol (X) can be obtained by reacting the compound of formula (IX) with a Grignard reagent. The C₁₅-salt of formula (XI) can be obtained from vinylionol (X) in the presence of a phosphine. A suitable method of obtaining compound (XI) is disclosed in WO2005/058811A1.

Vitamin A acetate (XIII) can finally be obtained by subjecting the C₁₅-salt of formula (XI) to Wittig conditions in the presence of the acetate of formula (XII). Details of such a Wittig reaction are disclosed in WO2005058811A1.

In one additional embodiment of the invention, the isobutene prepared by the method according to this invention described above is used to produce diisobutene. Diisobutene (2,4,4-trimethyl-I-pentene and 2,4,4-trimethyl-2-pentene as the main components) is an important industrial chemical and an important intermediate product in the production of other major industrial compounds. Processes for preparing diisobutene are known and described inter alia in Baerns et. al. Technische Chemie, 1st edition, Wiley-VCH, Weinheim 2006. One well-known way is the acid-catalyzed dimerization of isobutene.

The object of providing an alternative, improved method for the production of diisobutene preferably from sources of renewable raw materials is achieved by a method of producing diisobutene comprising the steps a) preparation of isobutene by the method according to this invention; b) dimerization of isobutene into diisobutene; c) purifying the diisobutene.

In the following, “diisobutene” means diisobutene produced from isobutene prepared by the method according to this invention.

Another embodiment of the invention are polymers comprising diisobutene in its polymerized form. Preferred are copolymers comprising, in polymerized form, at least one unsaturated carboxylic acid as defined in formula CoC below and diisobutene.

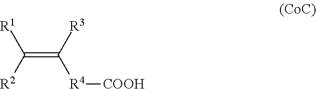

The variables in general formula (CoC) are defined as follows:
R¹, R² and R³ are independently selected from H, linear or branched C₁-C₁₂alkyl, linear or branched C₂-C₁₂alkenyl, wherein alkyl and/or alkenyl may be substituted with —NH₂, —OH, or —COOH; —COOH; and —COOR⁵, wherein R⁵ is selected from linear or branched C₁-C₁₂alkyl and linear or branched C₂-C₁₂ alkenyl.

R⁴ is selected from a single bond, —(CH₂)ₙ₁— with n1 being in the range of 0 to 4, —COO—(CH₂)ₖ— with k being in the range of 1 to 6, —C(O)—NH— and —C(O)—NR⁶—, wherein R⁶ is selected from linear or branched C₁-C₂₂alkyl, linear or branched C₂-C₂₂alkenyl, and C₆-C₂₂aryl.

Non-limiting examples of suitable unsaturated carboxylic acids include acrylic acid, methacrylic acid, 2-ethylacrylic acid, 2-phenylacrylic acid, malonic acid, crotonic acid, maleic acid (or maleic anhydride), fumaric acid, itaconic acid, aconitic acid, mesaconic acid, citraconic acid, sorbic acid, cinnamic acid, methylene malonic acid, unsaturated C₄-C₁₀dicarboxylic acids, and mixtures thereof.

One preferred copolymer consists of or comprises maleic acid and diisobutene in their polymerized forms. Another preferred copolymer consists of or comprises maleic anhydride and diisobutene in their polymerized forms.

Another preferred copolymer is the sodium salt of a copolymer consisting of or comprising, in their polymerized form, maleic acid or maleic anhydride and diisobutene, such copolymers having a K-value in the range of about 20 to about 80, preferably in the range of about 0 to about 50, more preferably in the range of about 35 to about 45, wherein the K-value is determined with about 1% dry substance in distilled water.

In one embodiment, the copolymer according to this invention consists of or comprises maleic acid or maleic anhydride and diisobutene in a weight ratio of about 1:1 and has a K-value of about 35.

In another embodiment, the copolymer consisting of or comprising maleic acid or maleic anhydride and diisobutene is modified by esterification of one carboxyl group of the polymerized maleic acid or maleic anhydride with oligo or polyalkoxylene compounds that may bear an alkyl end-capping, such end-capping preferably selected from with C₄ to C₁₈, C₆ to C₁₆, C₁₂ to C₁₄alkyl, whereas the other carboxyl-group of the polymerized maleic acid or maleic anhydride may be neutralized such that the copolymer may contain partially or—preferably-fully neutralized carboxyl groups.

In a further embodiment isoprenol obtained according to the processes described above is reacted with alkylenoxids to produce isoprenyl polyalkylene oxides. Preferably the isoprenyl polyalkylene oxides comprise 2 to 350, preferably 5 to 150 alkylenoxide repeating units. Preferred products are isoprenyl oxypolyethylene glycol and/or isoprenyl oxypolyethylene glycol C₁-C₄-alkyl ethers.

In one embodiment polymeric dispersants for inorganic binder compositions, preferably polycarboxylate ethers, are produced on the basis of isoprenyl polyalkylene oxides. In one embodiment the polymeric dispersant on the basis of isoprenyl polyalkylene oxides comprises at least one structural unit of the general formulae (XIVa), (XIVb), (XIVc) and/or (XIVd), wherein the structural units (XIVa), (XIVb), (XIVc) and (XIVd) can be the same or different both within individual polymer molecules and between different polymer molecules.

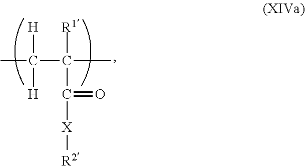

in which
R^{1′} is H or an unbranched or branched C₁-C₄alkyl group, CH₂COOH or CH₂CO—X—R^{3′}, preferably H or CH₃;

X is NH—(Cₙ*H₂ₙ*), O(Cₙ*H₂ₙ*) with n*=1, 2, 3 or 4, where the nitrogen atom or oxygen atom is attached to the CO group, or is a chemical bond, preferably X is a chemical bond or O(Cₙ*H₂ₙ*);
R^{2′} is OM, PO₃M₂, or O-PO₃M₂, with the proviso that X is a chemical bond, if R^{2′} is OM;

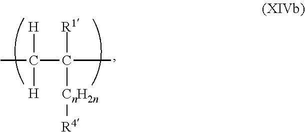

in which
R^{3′} is H or an unbranched or branched C₁-C₄alkyl group, preferably H or CH₃;
n is 0, 1, 2, 3 or 4, preferably 0 or 1;
R^{4′} is PO₃M₂, or O-PO₃M₂;

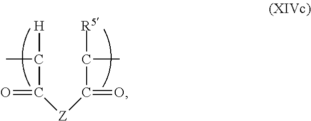

in which
R^{5′} is H or an unbranched or branched C₁-C₄alkyl group, preferably H; and
Z is O or NR^{7′}, preferably O;

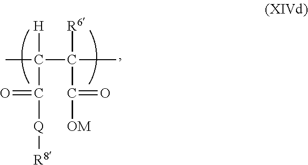

in which
R^{6′} is H or an unbranched or branched C₁-C₄alkyl group, preferably H;
Q is NR^{7′} or O, preferably O;
R^{7′} is H, (C_{n′}H_{2n′})—OH, (C_{n′}H_{2n′})—PO₃M₂, (C_{n′}H_{2n′})—OPO₃M₂, (C₆H₄)—PO₃M₂, or (C₆H₄)—OPO₃M₂,
R^{8′} is H, (C_{n″}H_{2n″})—OH, (C_{n″}H_{2n″})—PO₃M₂, (CₙH₂ₙ)—OPO₃M₂, (C₆H₄)—PO₃M₂, or (C₆H₄)—OPO₃M₂,
n′ is 1, 2, 3 or 4, preferably 1, 2 or 3;
n″ is 1, 2, 3 or 4, preferably 1, 2 or 3; and each M is independently of any other is H or a cation equivalent.

With particular preference, the structural unit of formula (XIVa) is a methacrylic acid or acrylic acid unit, the structural unit of formula (XIVc) is a maleic anhydride unit, and the structural unit of formula (XIVd) is a maleic acid or maleic monoester unit.

Where the monomers (XIV) are phosphoric esters or phosphonic esters, they may also include the corresponding diesters and triesters and also the monoester of diphosphoric acid. These esters come about in general during the esterification of organic alcohols with phosphoric acid, polyphosphoric acid, phosphorus oxides, phosphorus halides or phosphorus oxyhalides, and/or the corresponding phosphonic acid compounds, alongside the monoester, in different proportions, as for example 5-30 mol % of diester and 1-15 mol % of triester and also 2-20 mol % of the monoester of diphosphoric acid.

In a preferred embodiment the polymeric dispersant comprises at least one structural unit, based on the isoprenyl polyalkylene oxide, of the general formulae (XVa). All structural units XVa may be identical or different both within individual polyether side chains and between different polyether side chains.

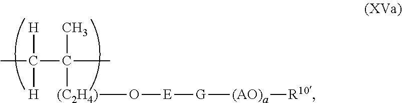

in which
E is an unbranched or branched C₁-C₆alkylene group, a cyclohexylene group, CH₂C₆H₁₀, 1,2-phenylene, 1,3-phenylene or 1,4-phenylene;
G is O, NH or CO—NH; or E and G together are a chemical bond;
A is CₓH₂ₓ with x=2, 3, 4 or 5, or is CH₂CH(C₆H₅), preferably x=2 or 3;
a is an integer from 2 to 350, preferably 5 to 150;
R^{10′} is H, an unbranched or branched C₁-C₄. alkyl group, CO—NH₂ and/or COCH₃.

Besides the structural units of the formulae (XIV) and (XVa), the polymeric dispersant may also comprise further structural units, which derive from radically polymerisable monomers, such as but not limited to hydroxyethyl (meth)acrylate, hydroxypropyl (meth)acrylate, (meth)acrylamide, (C₁-C₄)alkyl (meth)acrylates, styrene, styrenesulphonic acid, 2-acrylamido-2-methylpropanesulphonic acid, (meth)allylsulphonic acid, vinylsulphonic acid, vinyl acetate, acrolein, N-vinylformamide, vinylpyrrolidone, (meth)allyl alcohol, isoprenol, 1-butyl vinyl ether, isobutyl vinyl ether, aminopropyl vinyl ether, ethylene glycol monovinyl ether, 4-hydroxybutyl monovinyl ether, (meth)acrolein, crotonaldehyde, dibutyl maleate, dimethyl maleate, diethyl maleate and dipropyl maleate.

The average molecular weight Mw of the polymeric dispersant (comb polymer), as determined by gel permeation chromatography (GPC) is preferably 5000 to 200 000 g/mol, more preferably 10 000 to 80 000 g/mol, and very preferably 20 000 to 70 000 g/mol. The polymers were analysed for average molar mass and conversion by means of size exclusion chromatography (column combinations: OH-Pak SBG, OH-Pak SB 804 HQ and OH-Pak SB 802.5 HQ from Shodex, Japan; eluent: 80% by volume aqueous solution of HCO₂NH₄ (0.05 mol/l) and 20% by volume of acetonitrile; injection volume 100 μl; flow rate 0.5 ml/min). Calibration for determining the average molar mass is carried out using linear poly(ethylene oxide) and polyethylene glycol standards. The measure of the conversion is the peak of the copolymer, standardised to a relative height of 1, and the height of the peak of the unreacted macromonomer/PEG-containing oligomer is used as a measure of the residual monomer content.

The polymeric dispersant preferably meets the requirements of the industrial standard EN 934-2 (February 2002).

The polymeric dispersants comprising the structural units (XIV) and (XVa) are prepared in a conventional way, by means of radical polymerisation, for example. This is described for example in EP0894811, EP1851256, EP2463314, EP0753488.

In one additional embodiment of the invention, the isobutene, prepared by the method according to this invention described above, preferably from sources of renewable raw materials; is used as raw material for isobutene copolymers with ethylenically unsaturated mono and/or carboxylic acids and/or their esters, such as, for example, (meth)acrylates; with styrene and/or (meth)acrylonitrile. Such isobutene copolymers may be reacted, for example, to free-radical initiated aqueous emulsion polymers or suspension polymers, such as for example, for coating applications. Reference is made, for example, to U.S. Pat. No. 7,816,441, CA2667875 and EP450452. Such copolymers may be used as thermoplastic material, or when containing hydroxy functions, in two-component systems as a component reacting with hydroxyl group reactive cross-linkers, such as, for example, (blocked) polyisocyanates or amino resins (see, for example, to EP450452). In addition, copolymers of isobutene, prepared by the method according to this invention described above, preferably from sources of renewable raw materials; with maleimide may be used for coating applications (see, for example, U.S. Pat. No. 5,580,928).

### EXAMPLES

To illustrate the invention, various catalyst samples were synthesized and tested in a laboratory catalysis apparatus under the process conditions of a Guerbet reaction, with the condensation of methanol/ethanol in the gas phase to form i-butanol being examined. Precipitation processes and impregnation processes were used to synthesize the catalyst. A summary of the individual illustrative samples and their chemical composition is given in Table 1.

### Production of the Illustrative Samples

### Process by Means of Compacting and Impregnation

A commercially available finely divided hydrotalcite powder was firstly processed in a granulator (in the present case a roller compactor) to give a coarse-grained granular material. The coarse-grained granular material was rubbed through a sieve having a mesh opening of 1 mm and finely divided material was sieved out (500 μm mesh opening), giving a compacted hydrotalcite powder having a particle size in the range 0.5-1 mm. Pural® MG 70 (from Sasol) was used as commercially available hydrotalcite. Pural® MG 70 is aluminum magnesium hydroxy carbonate. It has a 70:30 MgO:Al₂O₃ ratio. A roller compactor from Powtec (model RC 100×30) was used as granulator. In the compaction operation, 250 g of finely divided powder were treated in a plurality of passes using corrugated rollers and a pressing pressure of 250 bar in each case. The rollers had a rotational speed of 5 rpm. The granulator was equipped with a sieve insert having a mesh opening of 1.6 mm.

The compacted hydrotalcite powder had a water absorption capacity (or liquid absorption capacity) of 0.46 ml/g and the loss on ignition of the compacted hydrotalcite powder, which was determined at 600° C., was 44.7% by weight. From one batch to the next, the water absorption capacity and the loss on ignition of the sample produced in each case could be subject to small deviations, which was then taken into account in the appropriate way for the addition of impregnation solution.

The application of the promoter elements to the compacted hydrotalcite powder was effected by means of impregnation. Firstly, 12 g each of the compacted hydrotalcite powder were placed in individual porcelain dishes having a diameter of 8 cm. The metal salt solutions were then added in the previously determined concentrations and amounts of liquid to the respective porcelain dishes in order to apply the desired target amount of metal species and not to exceed the liquid absorption capacity of the powder. This impregnation process on the dry powder is an incipient wetness process with complete filling of the pores. The porcelain dishes filled with powder samples were kept in motion or rotated during and after addition of the impregnation solution by means of a laboratory shaking machine, namely at a speed of 1000 rpm.

### Illustrative Sample B1

In the production of illustrative sample B1, a solution comprising Cu(NO₃)₂ 2.5H2O was used for application of the promoter. To produce the impregnation solution, 0.03 ml of Cu(NO₃)₂ 2.5 H₂O solution (admixed with 5.387 g of water were mixed and added to the initially charged 8 g hydrotalcite sample. To effect mixing or aging of the impregnated hydrotalcite sample, the porcelain dish filled with hydrotalcite sample was kept in motion for another 30 minutes at room temperature. The illustrative samples B2 to B7 shown in Table 1 were produced by the same method, with the promoter component(s) being varied in terms of composition.

To dry the sample, the porcelain dish charged with the sample was firstly stored at 80° C. in a convection drying oven for 16 hours. The sample was subsequently calcined in a calcination furnace (model LH120/12 from Nabertherm). For the calcination, the sample in the furnace was firstly heated to 250° C. and maintained at 250° C. for 4 hours. A stream of air of 6 l/min was introduced into the furnace during the entire calcination phase and cooling phase. In the heating phases, a heating rate of 5 K/min was used.

The calcined sample material was subjected to sieving in order to obtain the sample having a particle size of 0.5-1 mm. For this purpose, sieves having a mesh opening of 1 mm and 0.5 mm were used and the powder samples were firstly distributed over the area of the coarse sieve using the edge of a spatula and the fines were subsequently removed through the fine sieve.

TABLE 1
Summary overview of the illustrative samples which were used for
the catalytic tests, and also their chemical composition in respective
of the promoter elements and the starting materials used for the
synthesis and the basis of the support oxide. The abbreviation
PMG 70 means that Pural ® MG70 from Sasol was used for
synthesizing the catalyst. The samples were all calcined at 250° C.

| | | Cu | Ru | Ir | |
|---|---|---|---|---|---|
| | Catalyst | [% by | [% by | [% by | Basis of the |
| | No. | weight] | weight] | weight] | support oxide |
| 1 | B1 | 0.1 | 0 | 0 | PMG 70 |
| 2 | B2 | 1.0 | 0 | 0 | PMG 70 |
| 3 | B3* | 10.0 | 0 | 0 | PMG 70 |
| 4 | B4 | 5 | 0.1 | 0 | PMG 70 |
| 5 | B5 | 5 | 0 | 0.1 | PMG 70 |
| 6 | B6* | 0 | 0.1 | 0 | PMG 70 |
| 7 | B7* | 0 | 0 | 0.1 | PMG 70 |

*Comparative catalyst.

### Catalytic Studies

### Condensation of Different Alcohol Components (Methanol/Ethanol)

The results of the catalytic tests for the condensation of different alcohol components preferentially condensation of ethanol and methanol are shown in tables 2 to 5. The catalytic tests were, with only a few exceptions, in each case carried out on 0.5 and 1 ml of the pulverulent illustrative samples, using a crushed material fraction having a particle size in the range from 0.5 to 1 mm for this purpose. To prepare for the studies, the samples were positioned on a catalyst support grid or on a bed of inert particles in tube reactors, the loaded reactors were installed in 16-fold high throughput catalysis testing apparatus and the samples comprised therein were subject to the test procedures. The test procedures generally provided for the samples to be subjected to activation before the catalysis experiments. Activation of catalysts was performed at 400° C. under 20 vol % H₂ in inert gas for 10 h. In addition, all catalysts were also subjected to a conditioning treatment at 250° C. under the flow of 1 vol % ethanol for 24 h. All catalysis experiments were carried out at temperatures within the range from 250 to 325° C. A gas chromatograph coupled with a mass spectrometer (a GC-MS from Agilent) and equipped with FID and TCD was used for analyzing the product gas stream.

Another important finding was that the catalytic properties of the catalysts in respect of the process of the invention for condensing alcohols are significantly improved when the process is preceded by an activation and/or conditioning process.

An overview of a first series of experiments and the results achieved therein is shown in table 2. In the experiments shown there, the feed gas flow was selected so that the GHSV was 1000 h⁻¹.

An inert gas stream composed of nitrogen and argon loaded with ethanol, and methanol vapor was used as feed gas stream. The proportion of argon was kept constant in all experiments and was 5% by volume. The ethanol content was in each case set in the various experiments and was varied from 1 vol % to 20 vol % whereas methanol content was kept constant at 20 vol %.

Table 2 shows the results of the catalytic tests on the reaction of EtOH and MeOH at a reaction temperature of 325° C. using a feed comprising the EtOH and MeOH in a molar ratio of 1:20 in the presence of 20% by volume of H₂ at GHSV 1000 h⁻¹ at 7 bar reaction pressure.

| | | EtOH | MeOH | IsoBuOH | PrOH | Isobutanal | Propanal |
|---|---|---|---|---|---|---|---|
| | Catalyst | Conv (%) | Conv (%) | Sel (%) | Sel (%) | Sel(%) | Sel (%) |
| Example | | | | | | | |
| No. | | | | | | | |
| 1 | B1 | 90 | 25 | 41 | 3 | 2 | 0 |
| 2 | B2 | 100 | 57 | 24 | 0 | 10 | 0 |
| 3 | B4 | 100 | 73 | 13 | 1 | 5 | 0 |
| 4 | B5 | 100 | 63 | 20 | 1 | 8 | 0 |
| Comp. Ex. | | | | | | | |
| 1 | B6 | 25 | 46 | 5 | 5 | 0 | 0 |
| 2 | B7 | 53 | 28 | 20 | 9 | 16 | 0 |
| 3 | B3 | 95 | 86 | 14 | 2 | 5 | 0 |

Table 3 shows the results of the catalytic tests on the reaction of EtOH and MeOH at a reaction temperature of 325° C. using a feed comprising the EtOH and MeOH in a molar ratio of 1:20 in the presence of 20% by volume of H₂ at GHSV 2000 h⁻¹ at 7 bar reaction pressure.

| | | EtOH | MeOH | IsoBuOH | PrOH | Isobutanal | Propanal |
|---|---|---|---|---|---|---|---|
| | Catalyst | Conv (%) | Conv (%) | Sel (%) | Sel (%) | Sel(%) | Sel (%) |
| Example | | | | | | | |
| No. | | | | | | | |
| 5 | B1 | 60 | 11 | 28 | 15 | 0 | 4 |
| 6 | B2 | 98 | 37 | 31 | 0 | 0 | 0 |
| 7 | B4 | 100 | 58 | 18 | 0 | 0 | 0 |
| 8 | B5 | 100 | 49 | 24 | 0 | 0 | 0 |
| Comp. Ex. | | | | | | | |
| 4 | B6 | 28 | 21 | 9 | 9 | 0 | 0 |
| 5 | B7 | 42 | 13 | 20 | 15 | 0 | 0 |
| 6 | B3 | 95 | 70 | 16 | 2 | 0 | 1 |

Table 4 shows the results of the catalytic tests on the reaction of EtOH and MeOH at a reaction temperature of 325° C. using a feed comprising the EtOH and MeOH in a molar ratio of 1:20 at GHSV 1000 h⁻¹ at 7 bar reaction pressure.

| | | EtOH | MeOH | IsoBuOH | PrOH | Isobutanal | Propanal |
|---|---|---|---|---|---|---|---|
| | Catalyst | Conv (%) | Conv (%) | Sel (%) | Sel (%) | Sel(%) | Sel (%) |
| Example | | | | | | | |
| No. | | | | | | | |
| 9 | B1 | 34 | 10 | 16 | 15 | 11 | 4 |
| 10 | B2 | 96 | 26 | 22 | 2 | 41 | 1 |
| 11 | B4 | 100 | 55 | 19 | 0 | 15 | 0 |
| 12 | B5 | 100 | 45 | 22 | 0 | 22 | 0 |
| Comp. Ex. | | | | | | | |
| 7 | B6 | 1 | 31 | 6 | 7 | 0 | 0 |
| 8 | B7 | 34 | 18 | 19 | 15 | 3 | 0 |
| 9 | B3 | 100 | 39 | 25 | 0 | 27 | 0 |

Table 5 shows the results of the catalytic tests on the reaction of EtOH and MeOH at a reaction temperature of 325° C. using a feed comprising the EtOH and MeOH in a molar ratio of 1:20 at GHSV 2000 h⁻¹ at 7 bar reaction pressure.

| | | EtOH | MeOH | IsoBuOH | PrOH | Isobutanal | Propanal |
|---|---|---|---|---|---|---|---|
| | Catalyst | Conv (%) | Conv (%) | Sel (%) | Sel (%) | Sel(%) | Sel (%) |
| Example | | | | | | | |
| No. | | | | | | | |
| 13 | B1 | 16 | 5 | 12 | 15 | 8 | 3 |
| 14 | B2 | 67 | 12 | 9 | 10 | 43 | 10 |
| 15 | B4 | 90 | 30 | 19 | 3 | 28 | 2 |
| 16 | B5 | 91 | 21 | 17 | 3 | 43 | 3 |
| Comp. Ex. | | | | | | | |
| 10 | B6 | 15 | 11 | 15 | 18 | 2 | 0 |
| 11 | B7 | 26 | 7 | 15 | 16 | 6 | 2 |
| 12 | B5 | 91 | 21 | 17 | 3 | 43 | 3 |

### Equilibration

The Cu catalysts with high Cu content (1.0 and 10% by weight) were equilibrated under mixed Guerbet conditions (temperature of 325° C. using a feed comprising the EtOH and MeOH in a molar ratio of 1:20 in the presence of 20% by volume of H₂ at 7 bar reaction pressure) over 100 h time on stream. The selectivity of Cu catalysts changed from CO/CO₂ to Guerbet intermediates such as C₃/C₄ aldehydes and C₃/C₄ alcohols. This equilibration phase also led to the complete disappearance of CO₂ from the product spectrum over time on stream.

For example, a catalyst containing 0.1% by weight copper was more selective to C₄/C₃ products at the beginning and a catalyst containing 1.0% by weight copper produced more CO/CO₂, as with time on stream, the catalyst containing 1.0% by weight copper showed improved selectivity almost the same as the catalyst containing 0.1% by weight copper and also good conversion of EtOH and MeOH.

In summary, the copper comprising catalysts of the present invention were identified to successfully catalyze mixed Guerbet condensation of ethanol and methanol to isobutanol. The catalysts exhibited approximately 75% selectivity (carbon based) to C—C coupling products of mixed Guerbet reaction producing mainly isobutanol, isobutyraldehyde, propanol and propionaldehyde. A 20:1 (MeOH:EtOH) ratio was suitable for highest isobutanol selectivity. With changing MeOH:EtOH ratio, the nature of product distribution could be changed to produce mixture product stream of C₃-C₄ alcohols and aldehydes. Another standout factor could be attributed to the positive effect of high reaction pressure (7 bar) compared to that of literature. The process of the present invention represents a sustainable method to produce renewable isobutanol.

1.-15. (canceled)

16. A catalyst for condensing alcohols, which comprises support material in contact with copper as promoter, wherein
a) the support material comprises hydrotalcite-like compounds,
b) the proportion of copper promoter is in the range of from 0.05 to 5.0% by weight,
c) the support material has an Mg/Al ratio in the range of from 90/10 to 70/30, where the ratio is based on the weight of the respective oxides, wherein the catalyst may optionally comprise at least one further promoter element from the group consisting of Pt, Rh, Ru, Pd, Co, Ni, Pd, Cu, Ag and Au and the content of further promoter is in the range 0.01 to 2% by weight, with the proviso that the amount of copper promoter is greater than the amount of further promoter.

17. The catalyst for condensing alcohols according to claim 16, wherein the catalyst comprises at least one further promoter element from the group consisting of Ru and Ir and the content of further promoter is in the range of from 0.01 to 2.0% by weight.

18. The catalyst for condensing alcohols according to claim 16, wherein the proportion of copper promoter is in the range of from 0.05 to 2.0% by weight.

19. The catalyst for condensing alcohols according to claim 16, wherein the proportion of copper promoter is in the range 0.05 to 5.0% and Ir or Ru are present as further promoter in an amount of 0.05 to 0.15% by weight.

20. The catalyst for condensing alcohols according to claim 16, where the catalyst is obtained by a process comprising the following steps:
d) support material comprising a hydrotalcite-like compound or precursor material of a hydrotalcite-like compound is brought into contact with a promoter source,
e) an intimate mixture of support material comprising a hydrotalcite-like compound or precursor material of a hydrotalcite-like compound and the promoter source is produced,
f) the intimate mixture of support material comprising a hydrotalcite-like compound or precursor material of a hydrotalcite-like compound and the promoter source is treated thermally, with the thermal treatment comprising a calcination process at a temperature in the range 200-1000° C.

21. The catalyst for condensing alcohols according to claim 20, wherein the calcination process comprises heating the catalyst at least two different temperature levels.

22. The catalyst for condensing alcohols according to claim 20, wherein the catalyst is subjected to an activation treatment at 350 to 450° C. under 15 to 25 vol % H₂ in inert gas for 1 to 30 h or to a conditioning treatment at 200 to 300° C. under the flow of 0.5 to 5 vol % ethanol for 12 to 48 h.

23. The catalyst for condensing alcohols according to claim 16, wherein the catalyst is subjected to an equilibration treatment under the conditions specified in claim 16 over 50 to 200 h time on stream.

24. A process for condensing alcohols, the process comprising bringing an alcohol-comprising feed gas stream comprising an alcoholic component selected from the group consisting of C₁-C₅alcohols into contact with the catalyst according to claim 16.

25. The process of claim 24, wherein
(i) the process temperature is in the range from 200 to 450° C., or
(ii) the process pressure is in the range 0.05 to 60 bar, or
(iii) the alcohol content of the feed (gas) stream is in the range 0.5 to 90% by volume, or (iv) the feed (gas) stream has a GHSV in the range 500-5000 h⁻¹.

26. The process for condensing alcohols according to claim 24, wherein the molar ratio of component 1 to component 2 is in the range from 15:1 to 20:1.

27. The process for condensing alcohols according to claim 24 wherein the alcohols in the feed stream are methanol (component 1) and ethanol (component 2).

28. The process for condensing alcohols according to claim 24, wherein the feed stream comprises, apart from the at least two alcoholic components, reactive gas, and optionally inert gas, such as, for example, nitrogen or argon.

29. The process for condensing alcohols according to claim 24, wherein the ratio of alcohol to reactive gas is in the range from 40:2.5 to 20:10.

30. The process for condensing alcohols according to claim 29, wherein the at least two alcoholic components are methanol and ethanol and the reactive gas is hydrogen.

31. The process for condensing alcohols according to claim 24, wherein the alcohol content of the feed fluid stream is ≥10% by volume.
